(19) United States
(12) Patent Application Publication
(10) Pub. No.: US 2013/0064726 A1
(43) Pub. Date: Mar. 14, 2013

(54) APPLIANCE FOR AT LEAST PARTIALLY STERILIZING A CONTAMINATED SURFACE

(71) Applicants: Gregor Morfill, Muenchen (DE); Tetsuji Shimizu, Garching (DE); Yang-Fang Li, Garching (DE); Takuya Urayama, Middlesex (GB); Julia Zimmermann, Muenchen (DE); Bernd Steffes, Garching (DE)

(72) Inventors: Gregor Morfill, Muenchen (DE); Tetsuji Shimizu, Garching (DE); Yang-Fang Li, Garching (DE); Takuya Urayama, Middlesex (GB); Julia Zimmermann, Muenchen (DE); Bernd Steffes, Garching (DE)

(73) Assignees: Adtec Europe Ltd., Middlesex (GB); Max-Planck-Gesellschaft zur Foerderung der Wissenschaften e.V., Muenchen (DE)

(21) Appl. No.: 13/698,280

(22) PCT Filed: May 19, 2011
(86) PCT No.: PCT/EP2011/002506

§ 371 Date: Nov. 15, 2012

(30) Foreign Application Priority Data
May 19, 2010 (EP) 10005236.4
Oct. 25, 2010 (EP) 10013940.1
Mar. 10, 2011 (EP) PCT/EP2011/001177

(51) Int. Cl. A61L 2/14; A61C 19/06; A61B 18/14

(52) U.S. Cl. CPC A61L 2/14; A61B 18/14; A61C 19/06
USPC 422/186.21; 604/23; 433/32

(57) ABSTRACT

The invention relates to an appliance (18) for at least partially disinfecting/sterilising a contaminated surface (21), wherein the appliance (18) comprises an integrated plasma source for at least partially disinfecting/sterilising the surface by generating a non-thermal atmospheric plasma on the surface thereby reducing the concentration of pathogenic germs on the surface.

### FIELD OF THE INVENTION

The invention relates to an appliance, for at least partially sterilizing and/or disinfecting and/or decontaminating a contaminated surface.

### BACKGROUND OF THE INVENTION

The use of non-equilibrium plasmas (often referred to as non-thermal plasmas, low-temperature plasmas or cold atmospheric plasmas) for the in vivo sterilization of wounds is disclosed, for example, in U.S. Pat. No. 7,683,342 B2. However, the plasma source disclosed in this patent is not suitable for the regular sterilization and/or treatment of surfaces of appliances, e.g. kitchen appliances or laboratory tables, body surfaces or other surfaces under normal operating conditions of the appliance, i.e. during daily use of the appliance.

### SUMMARY OF THE INVENTION

It is therefore an object of the invention to improve the sterilization and/or disinfection and/or decontamination and/or treatment of surfaces of appliances, particularly kitchen appliances or laboratory tables, body surfaces or other surfaces.

This problem is solved by the idea to integrate a plasma source into an appliance, e.g. kitchen appliance or a laboratory table, wherein the integrated plasma source at least partially disinfects/sterilizes a surface by generating a non-thermal plasma on the surface thereby reducing the concentration of pathogenic germs on the surface.

In a preferred embodiment of the invention, the surface to be disinfected/sterilized is a surface of the appliance which is contaminated during use of the appliance. For example, kitchen appliances, e.g. work benches, generally comprise work plates or cutting boards, which are contaminated during the preparation of food. In other words, the surface to be disinfected/sterilized is preferably a part of the appliance which also includes the plasma source.

However, it is alternatively possible that the surface to be disinfected/sterilized is separated from the appliance including the plasma source. For example, the invention also encompasses a deodorant device for deodorizing a body surface, particularly in the form of a roll-on applicator or a spray device. In this embodiment, the plasma source is integrated into the deodorant device while the surface to be disinfected/sterilized is a body surface which is separated from the deodorant device.

In a preferred embodiment of the invention, the plasma source is a surface micro-discharge plasma source comprising several electrodes, wherein the surface micro-discharge plasma source generates micro-discharges on the surface of the plasma source. The basic principles of high-pressure plasma micro-discharges are explained, for example, in Hippler/Kersten/Schmidt/Schoenbach: “Low temperature plasmas”, Second Edition, Wiley Publishing House, Chapter 17. Therefore, reference is made to the afore-mentioned publication with regard to the basic principles of surface micro-discharge plasma sources, so that the afore-mentioned publication is incorporated by reference herein. However, it should be briefly mentioned that the surface micro-discharge plasma source comprises several electrodes which are spaced apart.

It should further be mentioned that there can be a uniform distance between the adjacent electrodes of different polarity. However, it is alternatively possible that there is spatially variable distance between the adjacent electrodes of different polarity.

Further, it should be noted that the surface micro-discharge plasma source is preferably embedded into a surface of the appliance, so that the non-thermal plasma is generated on top of the surface of the appliance. For example, the plasma source can be embedded in the surface of a work plate of a kitchen table so that the low-temperature plasma is generated on the surface of the work plate of the kitchen table thereby at least partially sterilizing the surface of the work plate.

It should further be noted that the embedded plasma source is preferably embedded in such a way that it is substantially flush with the surface of the appliance. Therefore, the plasma source preferably comprises a substantially plane surface which is flush with the plane surface of the appliance, e.g. a work plate of a kitchen table.

However, the invention is not restricted to appliances comprising a plane plasma source. It is also possible that the electrodes of the surface micro-discharge plasma source have a shape which resembles the shape of the surface of the appliance. For example, the plasma source can be integrated in a curved surface of the appliance so that the invention does not restrict the freedom of design of the appliance.

Further, there is variety of different arrangements of the electrodes of the surface micro-discharge plasma source.

In one embodiment of the invention, the electrodes of the surface micro-discharge plasma source are arranged in the same plane. For example, the electrodes can be finger-shaped intertwining each other from opposite directions. In another embodiment, the electrodes are spiral-shaped intertwining each other. Further, the electrodes can comprise interlocking branches or kinks.

In another embodiment of the invention, the electrodes are accessed sequentially by switching of the grounded part with a “cycling frequency” fC, thus enabling a propagating plasma source across the device. For instance, a “switched self sterilizing surface device” (100×50 cm2) with parallel electrodes (see FIG. 3C) separated by 5 mm operated with a cycling frequency of 1 Hz would have plasma production along a strip of length 50 cm for a time tp=10 ms. For a HV frequency fHV=2 kHz this would imply fHV.tP 20 bursts of surface micro discharges, enough to start the ion-molecule reaction chain. The plasma afterglow lasts for more than 1 s, so that the next cycle would enhance the local plasma chemistry. Such a device (100×50 cm2) could be operated with a power of 25W.

In another embodiment of the invention, the electrodes of the micro-discharge plasma source are not arranged in the same plane but in separate adjacent electrode layers, wherein each of the electrode layers is preferably planar and the separate electrode layers are preferably arranged coplanar relative to each other.

Moreover, it should be noted that the electrode arrangement of the plasma source is freely scaleable.

Further, it should be mentioned that the appliance according to the invention is preferably water-proof, dust-proof, air-born particles proof and/or easy to clean. This is particularly advantageous in case of a kitchen table comprising an integrated plasma source for disinfecting the work-plate of the kitchen table.

Moreover, the surface of the appliance comprising the integrated plasma source preferably consists of a corrosion resistant material, particularly ceramics, glass or glass-ceramics. Also, flexible corrosion resistant materials are preferred, such as e.g. silicone, Makro-Ion, and/or POM (polyoxymethylene).

It has already been mentioned that the appliance according to the invention can be a work plate, particularly on a kitchen table or on a laboratory table, or a cutting board for cutting objects, particularly food stuffs.

However, the plasma source according to the invention can alternatively be integrated into a handle, particularly a door handle, wherein the integrated plasma source sterilizes the surface of the handle. In particular in this case, the energy for the generation of the plasma is preferably harvested from the movement of the handle, particularly by means of a piezo device, inductively, or in any other suitable manner.

In another embodiment of the invention, the appliance is a bathroom equipment, particularly a toilet seat, comprising an integrated plasma source for sterilizing the surface of the bathroom equipment.

Further, it has already been mentioned that the invention also encompasses a deodorant device for deodorizing and/or disinfecting a body surface particularly in the form of a roll-on applicator or a spray device. In this embodiment, the deodorant device comprises an integrated plasma source applying a non-thermal plasma to the body surface which is to be deodorized.

However, the body surface which is to be deodorized and/or disinfected and/or sterilized is in no way limited to the surface of the armpit. Instead, in a preferred embodiment the appliance is constructed to be used in the intimate area, the feet and/or other parts of human or animal boy.

Another application of the invention is the sterilization of a moving handrail of an escalator or a moving walkway. In this embodiment, the plasma source can be arranged stationary close to the surface of the handrail, so that the non-thermal plasma generated by the plasma source at least partially disinfects/sterilizes the surface of the handrail. Alternatively, the plasma source can also be integrated into the moving handrail, so that the plasma source moves with the hand-rail.

Further, the invention also encompasses an appliance in the form of gym equipment, particularly in the form of a bench or a seat of a training machine. In this embodiment, the plasma source is integrated into the gym equipment thereby sterilizing the bacteria produced and/or delivered by the sweat. The energy for plasma generation may preferably be harvested from a movement of the gym equipment during the training. Therefore, a piezo device, a dynamo or a device harvesting energy by means of induction may be used. However, any other means suitable to gain energy from a movement of the training machine or gym equipment may be utilized.

It should also be noted that in all embodiments of the invention the power supply of the integrated plasma source can be provided wireless by an integrated battery and/or accumulator which allows a mobile use of the device. Alternatively, the power supply of the plasma source can be provided by connection to the general mains. Further, it is possible that the appliance includes at least one energy-harvesting device, e.g. a piezo crystal. In general, all appliances or devices according to the invention may be embodied as mobile, built-in or stand-alone devices which may be mains-driven or mains-independent.

In all embodiments of the invention, the energy for the plasma generation may preferably be gained from a movement of the device or appliance, which may be a movement of the appliance as such or a movement of parts of the appliance relative to each other. Preferably, an energy harvesting device is used, such that the energy is gained by at least one piezo crystal, more general a piezo device, a device gaining energy by induction, or any other suitable device.

Further, the invention encompasses also a device for reducing itching caused by insect bite, particularly in the form of a stick comprising the plasma source. In this embodiment, the device applies a non-thermal plasma to the skin surface at an insect bite thereby reducing itching.

Further, the invention is also suitable for protection against athletes' foot and other fungal deceases, particularly in damp environments, particularly swimming pools and saunas.

Moreover, the invention also encompasses a device for reducing toothache. Particularly, the device is embodied to be a stick which is constructed to apply plasma to an aching region of the teeth. Thus, germs, bacteria, microorganisms or other particles contaminating the area can be inactivated and/or killed, such that the source for the toothache is eliminated thereby reducing the ache. Also, the invention is suitable for the prophylaxis and/or treatment of gingivitis, periodontitis or other diseases of the gingiva, as well as caries.

The invention also includes a device for reducing ache and/or treating infections in at least one body orifice, in particular ears and/or nose. Generally, ache is reducible and/or infections are treatable in all body orifices by means of the device. More particularly, the shape or geometry of the device may be adapted for the treatment of at least one particular body orifice.

Further, the invention is preferably embodied as a device for the treatment and/or healing of wounds. The device can be mobile or fixedly installed particularly in a medical practice. Wounds can be most effectively sterilized, disinfected or decontaminated by plasma such that healing can be strongly accelerated. Further, a plasma treatment of wounds is most effective as tetanus prophylaxis.

Moreover, the invention encompasses a device for the treatment or healing of skin irritations. Plasma is a suitable medium for treating optically visible or otherwise irritating skin irritations, in particular by eliminating contaminating particles in the affected skin area. Also, acne, herpes and other diseases of the skin are efficiently treatable by the invention. Plasma is suitable for treating the skin also insofar as there is a regenerative effect of the plasma on the skin. This effect is also important in conjunction with the deodorant device according to the invention.

Moreover, the invention is suitable for the disinfection of baby bottles, pacifiers, toys, dentures, tooth brushes, razors, shavers, combs or hair brushes. Therefore, the invention includes a device suitable for the treatment of at least one of these objects, particularly by having a plasma source adapted to the shape of the respective object.

However, the invention also includes at least one of a baby bottle, pacifier, toy, denture, tooth brushe, razor, shaver, comb, and hair brush, the at least one object including an appliance for plasma sterilizing a surface of the object and/or a surface to be treated or to come in contact by/with the object.

The invention also includes an appliance adapted to be or integrated into a shopping cart, in particular the handle of a shopping cart. Preferably, the surface of the handle is sterilizable, in particular before the cart is used by a new customer. Therefore, the appliance may comprise a switch for starting the plasma action. Preferably, the action terminates after a predetermined time which is sufficiently long to sterilize, disinfect and/or decontaminate the respective surface. In a preferred embodiment, the energy for the plasma source may be harvested from the movement of the shopping cart, e.g. by means of a dynamo.

Another possible application of the invention is the use of a non-thermal plasma in a dishwasher or a dryer for sterilizing the dishes in the dishwasher or dryer.

Further, the concept of the invention can be applied in devices for disinfection of medical equipment or in the food industry for disinfecting objects.

In another embodiment, the appliance according to the invention comprises a conveyor belt, wherein the plasma source is arranged in the vicinity of the conveyor belt so that the plasma sterilizes objects conveyed on the conveyor belt. For example, the plasma source can be arranged beneath the conveyor belt so that the plasma is applied through the belt, which therefore has to be permeable for the plasma. Alternatively, the plasma source can be arranged stationary above or in the vicinity of the conveyor belt so that the plasma generated by the plasma source reaches the objects on the conveyor belt. Further, it is alternatively possible to integrate the plasma source into the conveyor belt.

The invention also encompasses an appliance which is built to be a container or which is adapted to have the inside of a container sterilized. Therefore, the appliance can be the container itself, part of the container or external to the container. In all cases the appliance is constructed to generate plasma inside the container in order to sterilize the inside and in particular an inner surface of the container. Moreover, the device can be adapted to sterilize and/or disinfect a bottle or a tube. Preferably, the bottle or the tube comprises a first electrode, wherein plasma can be generated inside the bottle or tube by the ignition of a discharge between the first electrode and the second electrode, the second electrode being integrated in an appliance external to the bottle or the tube. Alternatively, the bottle or tube may not comprise an electrode of its own, and plasma is generated inside the bottle or tube by bringing a plasma source close to or in contact to an outer wall segment or surface of the bottle or tube.

Further, the invention encompasses a device for disinfecting and/or sterilizing and/or decontaminating at least partially the udder or teats of a milkable animal. Preferably, the device can be integrated in a milking machine, which is most preferred when the device is applied to milk cows. Alternatively, the device can be embodied as a mobile device which is in particular preferable in conjunction with animals which are normally milked without using a milking machine. It is emphasized that the invention is usable with every milkable animal, e.g. cows, sheep, goat, buffaloes and many others.

It has already been mentioned that the invention also encompasses a deodorant device for deodorizing a body surface by applying a non-thermal plasma.

Preferably, the deodorant device comprises an applicator for applying or delivering a chemical agent. The chemical agent is preferably selected from at least one of a deodorant, an anti-transpirant and a fragrance. Most preferably, the applicator for the chemical agent is selected from at least one of a rotatable ball, a spray and a stick. Thus, the body part which is deodorized with the device is not only sterilized and/or disinfected and/or decontaminated, thereby removing the source of body malodour, but it is also possible to apply a chemical agent supporting the malodour preventing effect of the plasma and/or conferring an agreeable odour to the treated body part.

Throughout this application, the term “malodour” is meant to include all kinds of odour which should be masked, modified, prevented or reduced. Thus, it is not limited to unpleasant odours but includes odour which is as such not unpleasant, but shall nevertheless be masked, modified, prevented or reduced for any purpose.

In one embodiment, the deodorant device of the invention resembles the design of conventional deodorant devices comprising a rotatable ball. In this embodiment, the plasma source can be integrated into the rotatable ball, wherein the plasma source generates the plasma on the surface of the rotatable ball. Alternatively, the plasma source can be arranged stationary within the housing of the deodorant device but outside the rotatable ball. The rotatable ball serves as applicator for a chemical agent.

In a preferred embodiment of the deodorant device according to the invention, the deodorant device additionally comprises an applicator for applying a chemical agent, e.g. deodorant, to the skin surface wherein the agent applied to the skin surface interacts with the non-thermal plasma thereby improving the disinfecting/sterilizing effect of the non-thermal plasma. In other words, the non-thermal plasma applied by the plasma source and the chemical agent applied by the applicator interact with each other so that the disinfecting/sterilizing effect is enhanced by the interaction between the chemical agent and the non-thermal plasma.

In another embodiment, the deodorant device does not comprise any moveable parts and relies solely on the disinfecting/sterilizing effect of the plasma.

It should also be noted that the term “pathogenic germs” as used in this description encompasses bacteria, spores, viruses, fungi, prions, micro organisms and bio-films comprising any of the aforementioned pathogenic germs. Also included are allergens, and all other molecules causing any inconvenience, disturbance and/or debilitation. Further, air-borne particles are included, e.g. pollen.

The invention and its particular features and advantages will become more apparent from the following detailed description considered with reference to the accompanying drawings.

### BRIEF DESCRIPTION OF THE DRAWINGS

FIG. 1 shows an exploded perspective view of a plasma source according to the invention, which can be integrated into an appliance, e.g. a kitchen table.

FIG. 2 shows a perspective view of the plasma source according to FIG. 1.

FIGS. 3A-3I show different embodiments and views of a plasma source according to the invention, including the “switched self sterilizing surface” in FIG. 3C, wherein switches are closed and opened sequentially at a rate fC/n, with n being the number of grounded electrodes.

FIG. 4 shows a perspective view of a kitchen block comprising a self-sterilizing work plate.

FIG. 5 shows a perspective view of a laboratory table comprising a self-sterilizing work plate.

FIG. 6 shows a perspective view of a toilet seat comprising an integrated plasma source for sterilizing the toilet seat.

FIG. 7A shows a schematic view of an escalator comprising a plasma source for sterilizing the moving handrail of the escalator.

FIG. 7B shows a modification of the embodiment of FIG. 7A wherein the plasma source is integrated into the moving handrail.

FIG. 8 shows a schematic view of the deodorant device comprising an integrated plasma source.

FIG. 9 shows a modification of the embodiment of FIG. 8 additionally comprising nozzles for applying a chemical agent onto the body surface.

FIG. 10 is a modification of the embodiment of FIG. 8 comprising a rotatable ball wherein the plasma source is integrated into the rotatable ball.

FIG. 11 shows a side view of another embodiment of a deodorant device.

FIG. 12 shows a front view of the deodorant device depicted in FIG. 11.

FIG. 13 shows a longitudinal cut through the front end of the deodorant device according to FIGS. 11 and 12.

FIG. 14 shows a perspective view of another example of a deodorant device comprising an integrated plasma source.

FIG. 15 shows a schematic view of another example of a deodorant device comprising an integrated plasma source.

FIG. 16 shows a schematic view of an example of a device suitable in particular for reducing toothache and treatment or healing of skin irritations as well as for reducing itching caused by insect bites.

FIG. 17 shows a schematic view of a device suitable for sterilizing and/or disinfecting at least partially the udder and/or teats of a milkable animal.

FIG. 18 shows a schematic view of a washing machine comprising an integrated plasma source.

FIG. 19 shows a simplified side view of a conveyor comprising a plasma source for sterilizing objects on the conveyor.

FIG. 20 is a diagram showing the switching of the plasma source in the appliance according to the invention.

FIG. 21 shows a schematic cross section through a container.

FIG. 22 shows a top view onto a lid 89 of the container depicted in FIG. 21.

FIG. 23 shows an enlarged cross section through the lid 89.

FIG. 24 shows a top view of a counter electrode 91 of the container shown in FIG. 21.

FIG. 25 shows a lid (left-hand) and a counter electrode (right-hand) which are used in case the lid 89 is soft and bendable.

FIG. 26 shows another embodiment of a lid (left-hand) and a counter electrode (right-hand).

FIG. 27 shows a schematic view of another example of an appliance for sterilizing the inside of a container.

FIG. 28 shows yet another example of an appliance for sterilizing the inside of a container in a schematic view.

FIG. 29 shows an appliance for sterilizing the inside of a bottle in a schematic view.

FIG. 30 shows a schematic view of a first example of a bottle comprising a first electrode.

FIG. 31 shows a second example of a bottle comprising a first electrode in schematic view.

FIG. 32 shows a third example of a bottle comprising a first electrode in a schematic view.

### DETAILED DESCRIPTION OF THE DRAWINGS

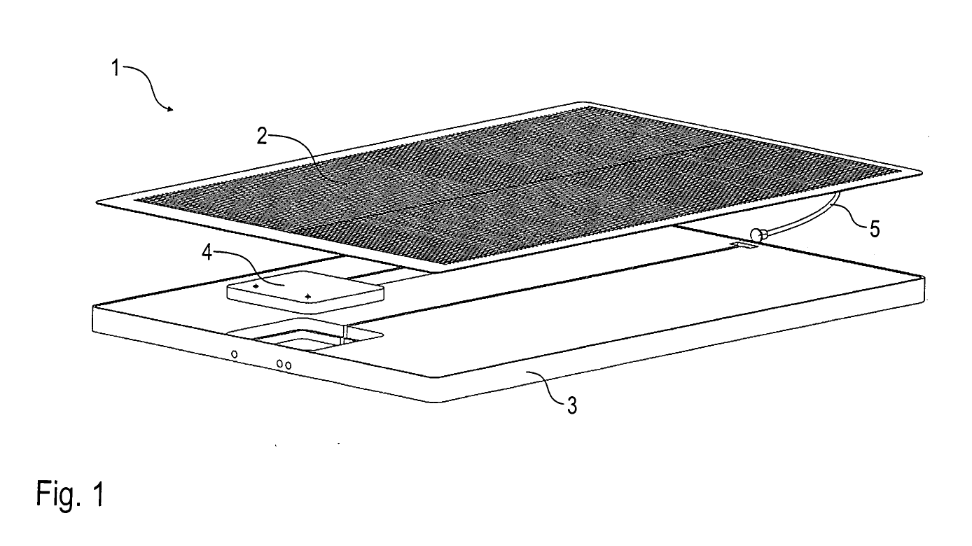

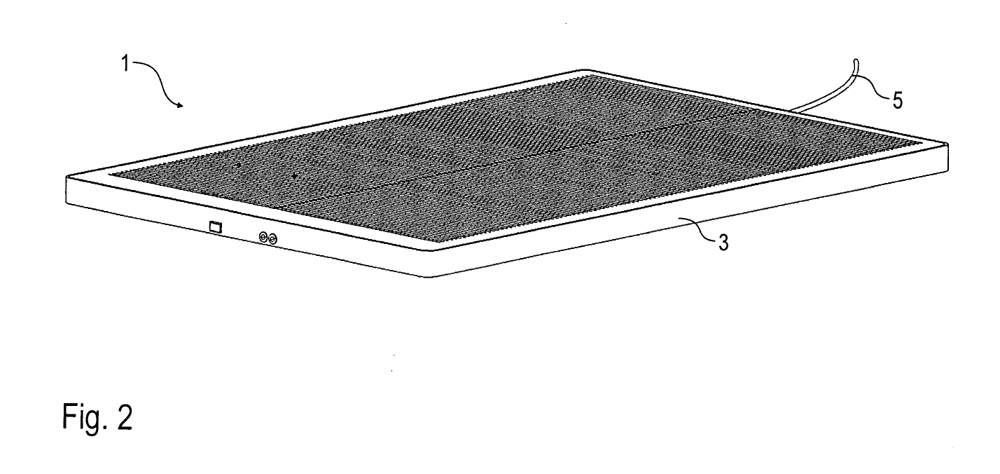

FIGS. 1 and 2 illustrate an embodiment of a plasma source 1 which can be integrated into an appliance, e.g. a work plate of a kitchen table, which will be described in more detail later.

The plasma source 1 comprises a flat and planar electrode arrangement 2, a housing 3, a driver circuit 4 for driving the electrode arrangement 2 and a connection cable 5 for connecting the plasma source 1 to mains.

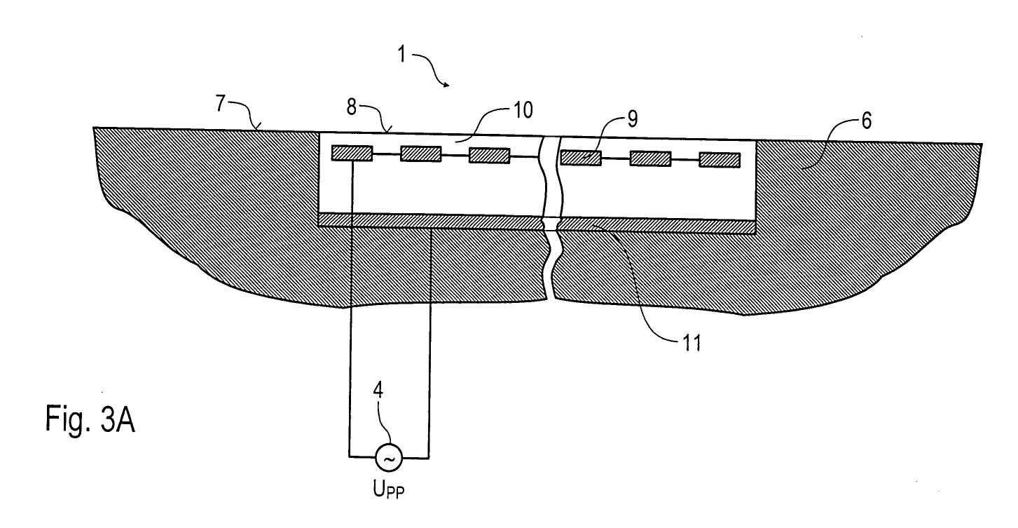

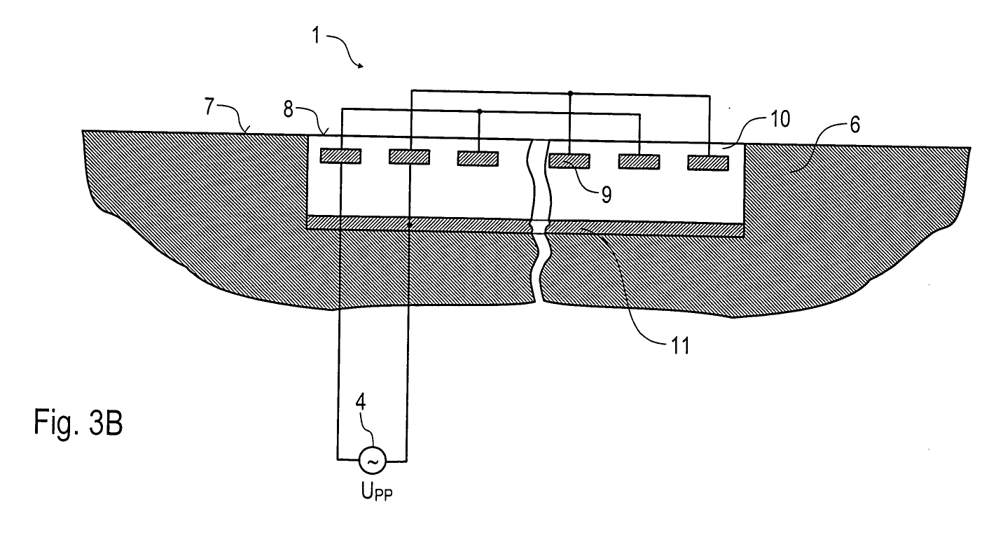

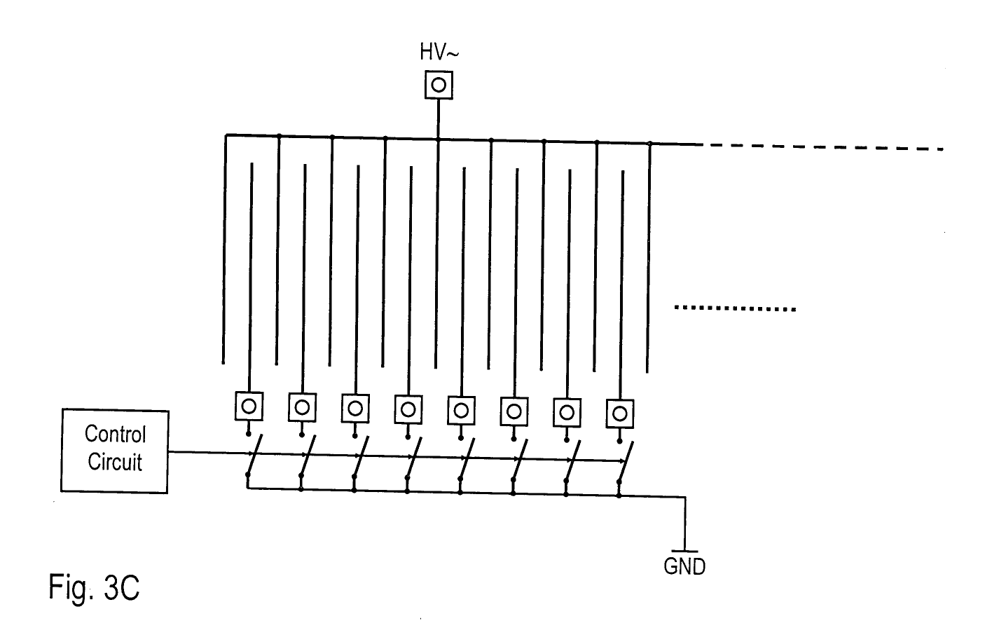

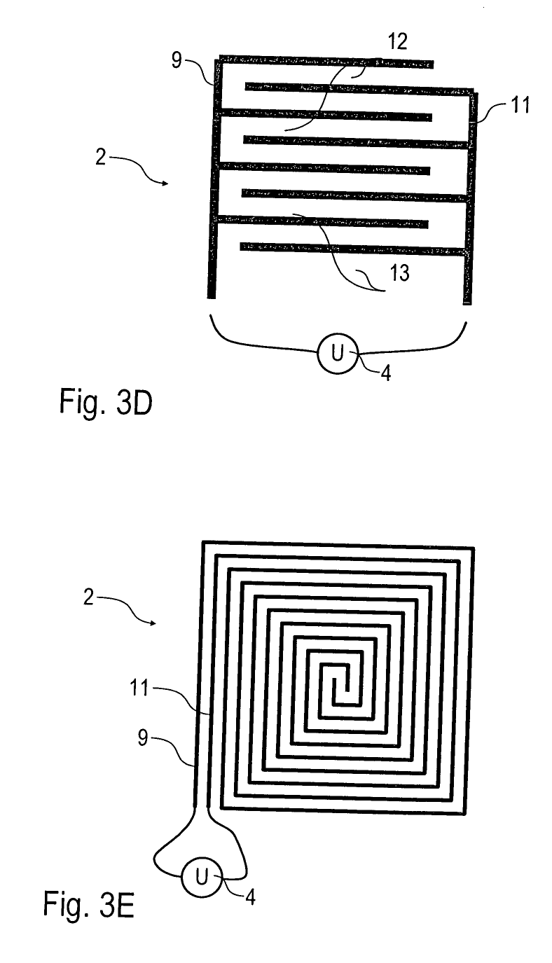

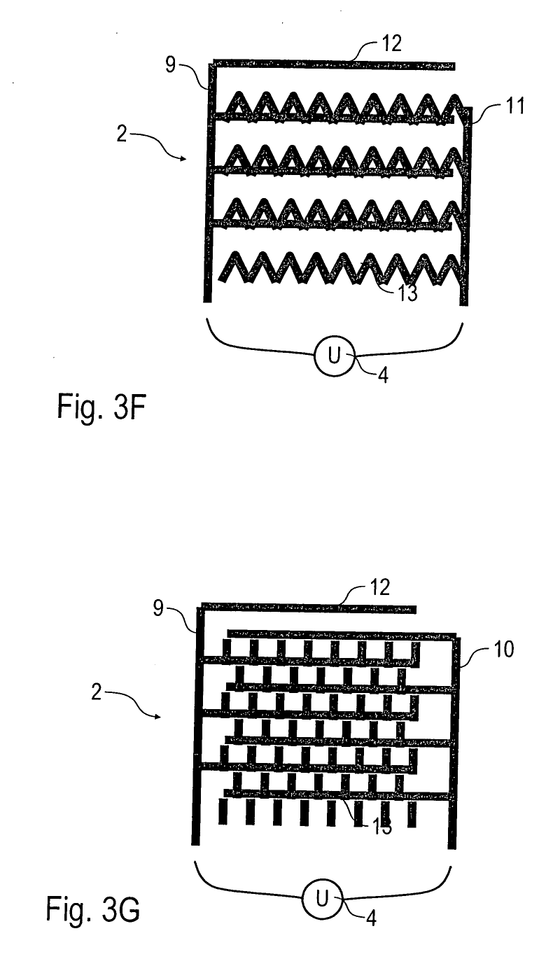

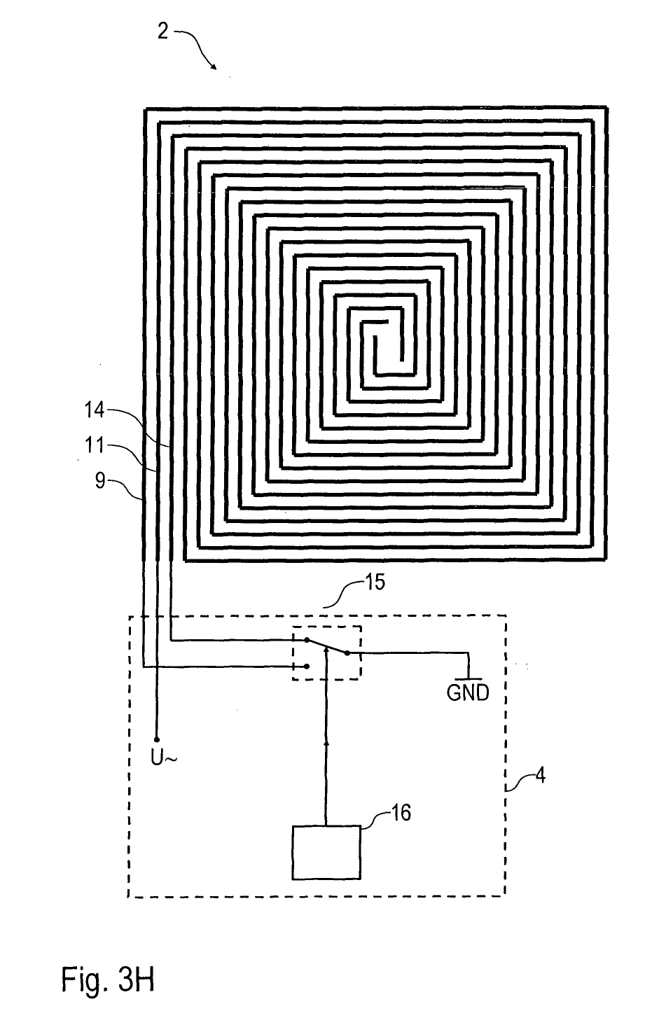

FIG. 3A shows a simplified cross section of a first embodiment of the electrode arrangement 2 of the plasma source 1.

Firstly, it should be noted that the plasma source 1 is integrated into an open cavity of an appliance 6 so that the appliance 6 and the plasma source 1 comprise surfaces 7, 8 which are flush so that the surface 8 of the plasma source 1 constitutes a part of the surface 7 of the appliance 6.

Further, it should be noted that the electrode arrangement 2 of the plasma source comprises several grid-shaped electrodes 9 which are interconnected with each other and embedded into a dielectric layer 10. Further, the electrode arrangement 2 comprises a common back electrode 11 at the under side of the dielectric layer 10.

During operation, the driver circuit 4 applies a AC voltage U to the electrodes 9, 11 so that surface micro-discharges are triggered on the surface 8 of the plasma source 1 as explained in the above-mentioned book titled “Low temperature plasmas”.

From FIG. 3A it is obvious that the electrode 9 is embedded in the dielectric layer 10. The plasma source 1 thus realizes the principle of a self-sterilizing surface (SSS). However, in another embodiment the first electrode 9 can form a part of the surface 8 or be located on the surface 8. In this case, the plasma source realizes in general the principle of a surface micro-discharge (SMD) source with an accessible or exposed electrode.

Yet another possibility is that the appliance includes only the electrode 11 with a dielectric layer 10 disposed thereon. In order to sterilize and/or disinfect and/or decontaminate a surface external to the appliance, the surface to be treated is used as a counter electrode to the electrode 11. Such a device realizes the principle of a dielectric barrier discharge (DBD) plasma source.

For the invention as explained here, the principle of a self-sterilizing surface (SSS) is most preferred. It should be emphasized that within this principle it is not only possible to sterilize the surface in which the electrode 9 is embedded, but it is also possible to sterilize an external surface which faces the surface 8. However, for the appliances as described herein, also a plasma source having an exposed or accessible electrode 9 (SMD) is preferred.

Last, the appliances may also include a dielectric barrier discharge (DBD) plasma source. Such an embodiment is still within the scope of the invention in spite of the fact that there are certain inconveniences in conjunction with such an embodiment. For example, if an appliance including a dielectric barrier discharge (DBD) plasma source is used with human skin as a counter electrode, a current will flow through the skin which will almost always not be agreeable.

In a preferred embodiment, the dielectric in which the electrode 9 is embedded in the case of a SSS plasma source is a solid phase material. However, it is emphasized that it does not need to be a solid phase material or a solid layer. Generally, in other preferred embodiments it is possible to embed the electrode 9 in viscous materials like cream, in particular vanishing cream, night cream, hand cream, lotion, or liquid materials like perfumes, fragrances, sterilizing and/or disinfecting liquids, cleaning agents, oil, or bulk material, in particular bulk material which is charged or loaded with a dielectric medium, e.g. zeolites. Further, at least one impregnated towelette or soaked cloth or other loadable/chargeable materials can be provided for carrying a dielectric medium. Preferably, the medium is chosen to comprise a suitable dielectric constant. It may comprise additives to further enhance the effect of the plasma and/or the medium.

Preferably, a lower electrode of the SSS source is covered by a first dielectric, which is normally a solid material. On top of this dielectric, an upper electrode is arranged which may be embedded in a non-solid dielectric material. In order to guarantee a suitable thickness of the non-solid dielectric, a suitable distance of the upper electrode to the surface to be treated, and a preferably homogeneous distribution of the non-solid dielectric on the upper electrode, there may be provided spacers, like bars or webs, preferably dividing the region of the upper electrode in separated chambers filled with the dielectric. Further, wires or meshes of the upper electrode may serve as spacers, in particular if they project beyond a surface of the non-solid dielectric, such that plasma is generated in the space between the treated surface and the surface of the dielectric, this space being defined by the height of the wires. In this latter case, however, a SMD source is realized instead of a SSS source.

When the plasma is generated, a non-solid dielectric medium and/or at least one additive comprised thereof is preferably evaporated and most preferably activated by the plasma. Thus, the medium and/or the additive is/are transported to the surface to be treated. Alternatively or in addition, the surface of the dielectric and the surface to be treated can be in contact, such that the non-solid dielectric can be directly applied to the treated surface, e.g. human skin.

FIG. 3B shows a modification of the plasma source 1 according to FIG. 3A, wherein this embodiment corresponds to the previous embodiment to a large extent. Therefore, reference is made to the above description of the embodiment according to FIG. 3A and the same reference numerals are used for corresponding parts and details.

One characteristic of this embodiment is the electrical connection of the upper electrodes 9 which are alternatively connected to the different poles of the driver circuit 4.

FIG. 3C shows another modification of the embodiment of FIG. 3A, wherein this embodiment corresponds to the previous embodiment to a large extent. The difference lies in the mode of operation, the switching of the discharge from one segment to the next. This technique makes use of the plasma afterglow and allows production of large self sterilizing surfaces with low energy requirements.

Therefore, reference is made to the above description with regard to FIG. 3A and the same reference numerals are used for corresponding parts and details.

One characteristic of this embodiment is that the back electrode 11 is missing.

It should further be noted that it is alternatively possible to switch the high voltage lines, as well. Further, it is possible to switch all electrode pairs successively pair by pair.

FIG. 3D shows an alternative design of the electrode arrangement 2, wherein the electrodes 9 and 11 each comprise electrode fingers 12, 13 intertwining each other. In this embodiment, the electrodes 9, 11 along with their electrode fingers 12, 13 are arranged in the same plane.

FIG. 3E shows another design of the electrode arrangement 2 wherein the electrodes 9, 11 are spiral-shaped intertwining each other.

FIG. 3F shows a modification of the electrode arrangement 2 of FIG. 3D, wherein this embodiment corresponds to the embodiment of FIG. 3D to a large extent. Therefore, reference is made to the above description with regard to FIG. 3D and the same reference numerals are used for corresponding parts and details.

One characteristic of this embodiment is that the electrode fingers 13 of the electrode 11 are staggered.

FIG. 3G shows another modification of the electrode arrangement, wherein the electrode fingers 13 of the electrode 11 comprises interlocking branches.

FIG. 3H shows a modification of the electrode arrangement 2 according to FIG. 3E, so that reference is made to the above description and the same reference numerals are used for corresponding parts and details.

One characteristic of this embodiment is that the electrode arrangement 2 comprises an additional electrode 14 besides the electrodes 9, 11. All the electrodes 9, 11, 14 are spiral-shaped intertwining each other.

Further, the driver circuit 4 comprises one switching element 15 connecting the electrodes 9 and 14 alternatively with ground GND, wherein the switching element 15 is controlled by a control device 16.

FIG. 31 shows a modification of the embodiment of FIG. 3H, wherein this embodiment corresponds to the previous embodiment to a large extent. Therefore, reference is made to the above description and the same reference numerals are used for corresponding parts and details.

One characteristic of this embodiment is that the spiral-shaped electrode 11 is staggered.

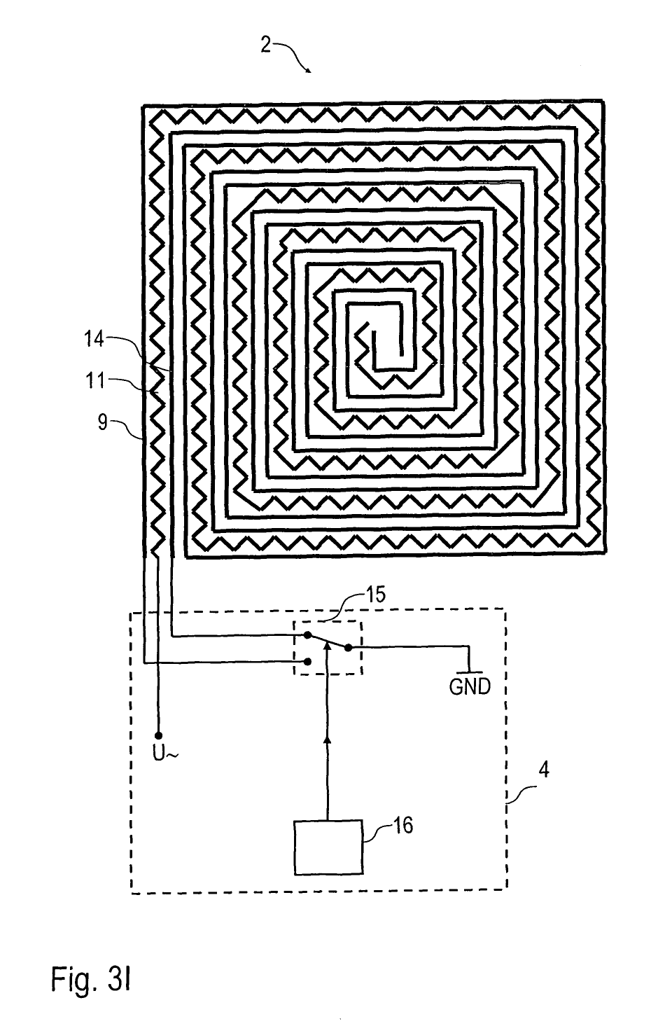

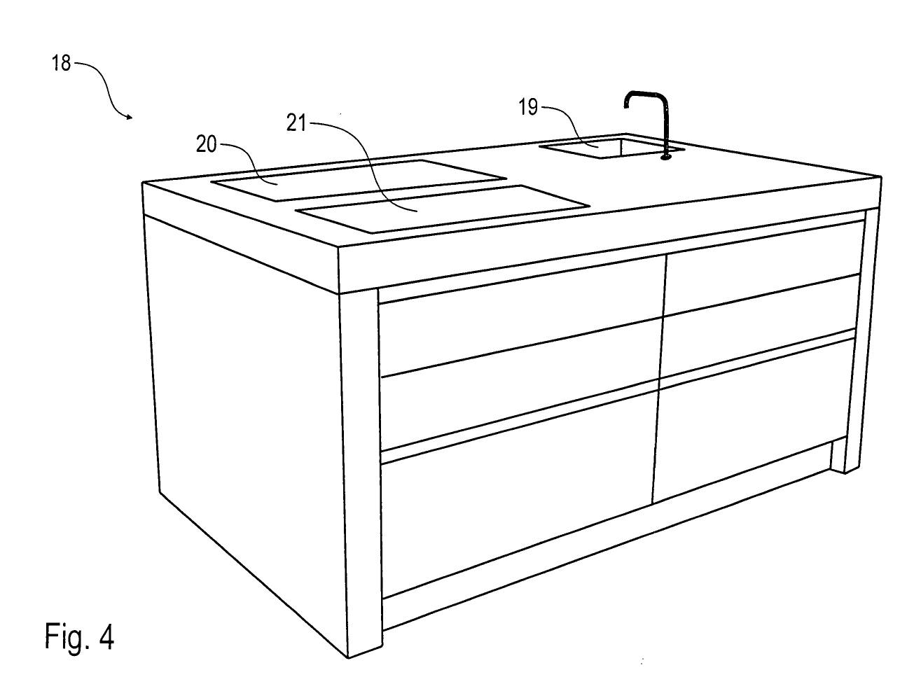

FIG. 4 shows a perspective view of a kitchen block 18 comprising a sink 19 and a ceramic stove top 20 which is per se known from the state of the art. However, the kitchen block 18 additionally comprises a self sterilizing work plate 21 comprising an integrated plasma source as mentioned above. The integrated plasma source generates a low-temperature plasma on the surface of the self-sterilizing work plate 21 thereby sterilizing the surface of the work plate 21 at least partially.

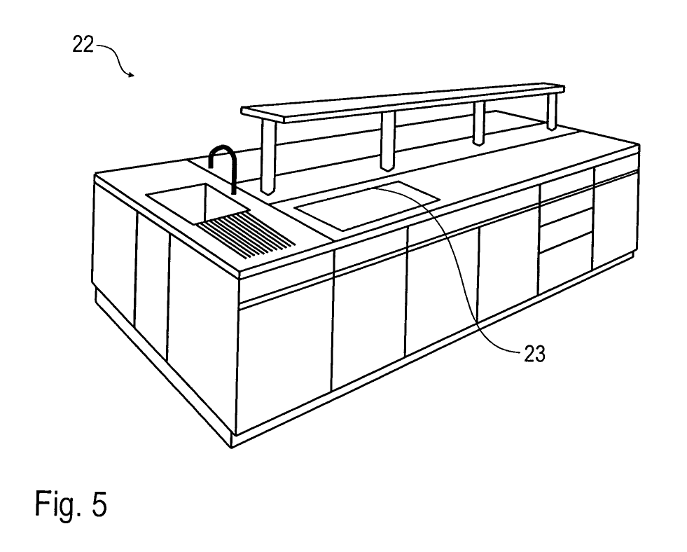

FIG. 5 shows a perspective view of a laboratory table 22 comprising a self-sterilizing work plate 23 similar to the self-sterilizing work plate 21 of the kitchen block 18 according to FIG. 4.

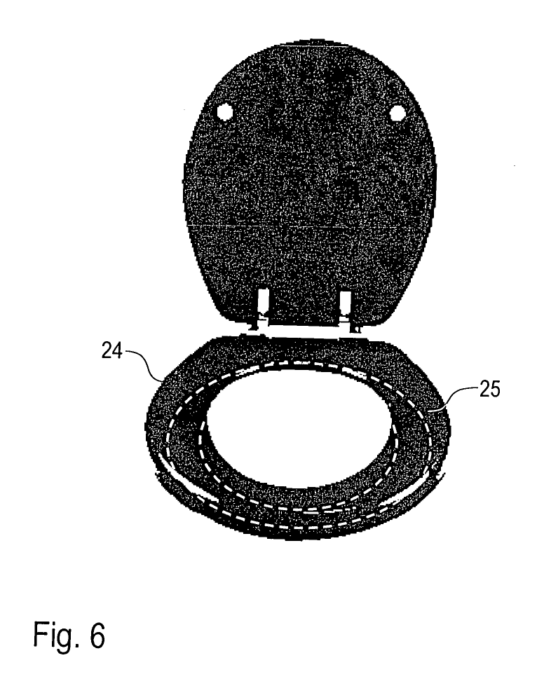

FIG. 6 shows a perspective view of a toilet seat 24 comprising an integrated plasma source 25 which is embedded into the toilet seat 24 and generates a low-temperature plasma on the surface of the toilet seat 24 thereby sterilizing the toilet seat at least partially.

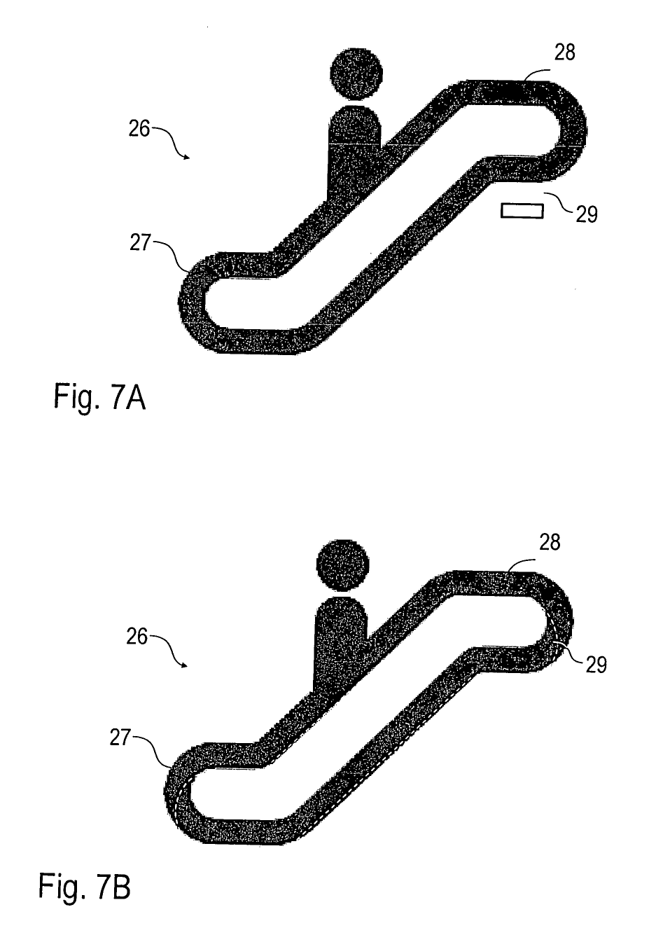

FIG. 7A shows a schematic view of an escalator 26 comprising moving handrails 27, wherein the surface 28 of the moving handrails 27 is sterilized by a stationary plasma source 29 which is arranged beneath the moving handrail 27. The plasma source 29 applies a low-temperature plasma to the surface 28 of the moving handrail 27 thereby sterilizing the surface 28.

FIG. 7B shows a modification of the embodiment of FIG. 7A, wherein this embodiment corresponds to the previous embodiments to a large extent. Therefore, reference is made to the above description and the same reference numerals are used for corresponding parts and details.

One characteristic of this embodiment is that the plasma source 29 is not stationary but arranged within the moving handrail 27 so that the plasma source 29 moves along with the moving handrail 27.

As already mentioned, in one preferred embodiment the appliance can be a deodorant device which is suitable for deodorizing and/or disinfecting a body surface. Mainly, deodorant devices are used to deodorize and/or disinfect the armpits. However, the invention is in no way limited to this body region. There is a preferred embodiment, wherein the appliance is constructed to deodorize, disinfect and/or sterilize the intimate area of the body. Yet another embodiment is constructed to sterilize the feet.

In this particular case, the appliance can be constructed to be a shoe or can be integrated into a shoe. Also, the appliance can be embodied to be a shoe tree or it can be integrated into a shoe tree. The same holds for an insole for a shoe.

Further, in a preferred embodiment, the appliance is integrated into clothing. Thus, a body part can be deodorized, disinfected and/or sterilized while wearing the clothing.

Moreover, since the plasma generated by the appliance is able to penetrate the fabric of textile materials like clothing, it is not only possible to apply the deodorant device or the appliance to the naked skin, but it is also possible to apply the plasma generated by the appliance through the clothing when dressed. Thus, it is not only possible to apply the deodorant device once in the morning after a general cleaning of the body, but it is also possible to deodorize, disinfect and/or sterilize a body surface during the course of the day without undressing. In particular, feet can be treated through stockings or socks.

In particular, when applying the plasma through the clothing, the clothing itself is also sterilized and/or disinfected and/or decontaminated. While the plasma penetrates the fabric of the textile material of the clothing, bacteria, germs and other particles causing malodour or contaminating the clothing are killed and/or inactivated, such that this source of malodour is drastically reduced.

The deodorant device 30 may preferably comprise an applicator for applying or delivering a chemical agent. The chemical agent is preferably selected from at least one of a deodorant, an anti-transpirant, a fragrance, a perfume, a bactericidal agent, an agent which is harmful to bacteria, fungi, spores, germs, viruses, prions, biofilms comprising any of the afore-mentioned or other pathogenic germs, and/or micro-organisms, an agent inhibiting bacterial growth or the growth of other contaminating particles or pathogenic germs, an agent generally suitable for skin care, particularly a moisturizing agent, a hair growing agent, a hair tonic, and a hair restorer.

The applicator for the chemical agent is preferably selected from at least one of a rotatable ball, a spray, and a stick.

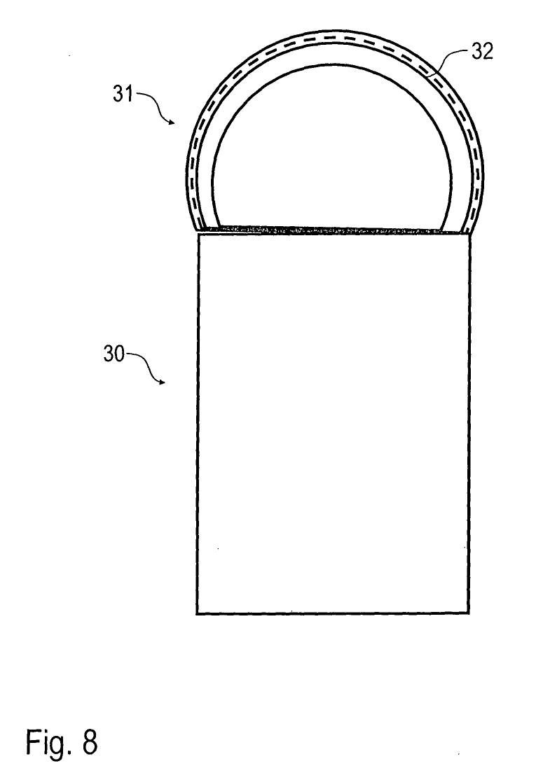

FIG. 8 shows a simplified side view of a first example of a deodorant device 30 comprising a ball-shaped head 31 with an integrated plasma source 32, wherein the plasma source 32 generates a low-temperature plasma on the surface of the ball-shaped head 31. The deodorant device 30 is used in the same way as conventional deo-rollers, i.e. the ball-shaped head 31 is moved over the body surface to be sterilized so that the low-temperature plasma generated on the surface of the ball-shaped head 31 sterilizes the body surface. According to the embodiment of FIG. 8, the ball-shaped head 31 is preferably not rotatable. This is insofar advantageous, as the plasma source can be most easily integrated in the ball-shaped head 31 and/or in the lower part of the deodorant device 30. In the latter case, preferably only the at least one electrode of the plasma source is integrated into the ball-shaped head 31, while the remaining parts of the source are integrated into the lower part of the deodorant device 30. Since the ball-shaped head 31 is not rotatable, there is no problem at all in contacting the at least one electrode with the remaining parts of the plasma source electrically. However, the surface on which the plasma is generated has a curved shape, in particularly convex within this embodiment. Moreover, it has preferably the shape of a spherical segment. This is favourable particularly in conjunction with surfaces to be sterilized having a curved shape of its own. Therefore, the embodiment depicted in FIG. 8 is in particular suitable for the treatment of human armpits, where the curved shape of the armpit surface should more or less follow the surface of the deodorant device 30 such that a preferred distance between the armpit surface and the surface on which the plasma is generated has an optimal value over a large region of the device on the one hand and the armpit on the other hand.

Preferably, the deodorant device 30 can be opened, most preferably swing opened. In a preferred embodiment, the ball-shaped head 31 can be swung away from the lower part of the device. By opening the deodorant device 30, a battery or another part of the plasma source can be changed or a reservoir of an applicator for a chemical agent can be refilled or changed.

In order to generate the plasma efficiently on a surface of the deodorant device 30, a certain distance between the surface to be treated and the surface on which the plasma is generated should be maintained. For this purpose, the deodorant device or any other appliance for treating a surface with plasma can include spacers such as knobs, ribs, meshes or other suitable structures, preferably including at least one projection on the surface on which the plasma is generated. However, in another embodiment, the surface can include recessed areas, in which the plasma is generated. The parts of the surface which are not recessed then form spacer elements which guarantee a certain distance to the surface to be treated, such that plasma can be generated efficiently.

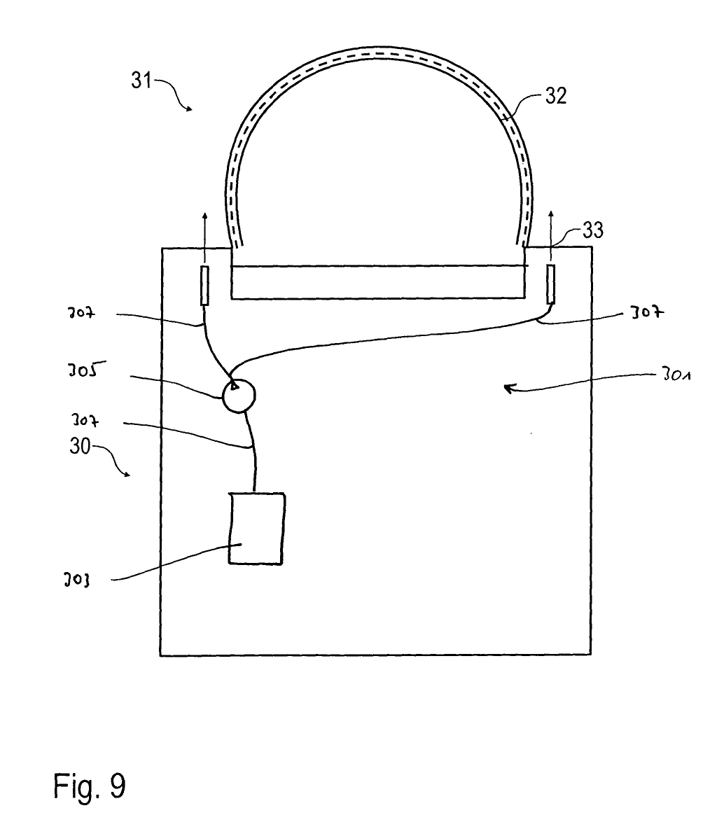

FIG. 9 shows a modification of the deodorant device 30 according to FIG. 8, wherein this embodiment corresponds to the previous embodiment to a large extent. Therefore, reference is made to the above description and the same reference numerals are used for corresponding parts and details.

One characteristic of this embodiment is that the deodorant device 30 additionally comprises nozzles 33 for applying a chemical agent onto the body surface to be sterilized. The chemical agent applied by the nozzles 33 then interacts with the low-temperature plasma thereby enhancing the sterilizing effect and/or masking the smell of the low-temperature plasma. The nozzles 33 are part of an applicator generally indicated as 301. The applicator 301 serves for applying or delivering the chemical agent and includes in this embodiment a reservoir 303, a pumping device 305 for pumping the agent from the reservoir 303 to the nozzles 33, and pipes or tubes 307 for guiding the agent from the reservoir 303 via the pumping device 305 to the nozzles 33. The pumping device 305 can be electrically driven, in particular mains-driven, battery-driven, driven by an energy-harvesting device or driven by an accumulator. However, in another embodiment, the pumping device 305 is part of a pump spray, such that the user of the deodorant device pumps the agent from the reservoir 303 to the nozzles 33 due to its own motion.

It is possible that generation and application of the chemical agent are carried out simultaneously. However, in another embodiment, plasma activation and application of the chemical agent can be controlled independently. In particular, it is possible to first sterilize and/or disinfect a body surface with plasma, and afterwards applying the chemical agent. In still another embodiment, the chemical agent can be applied first while the plasma acting on the chemical agent already applied to the skin changes the properties of the chemical agent, in particular by activating the agent and/or improving the effect of the agent, be it a bactericidal effect, a smell, an effect inhibiting bacterial growth, or another effect.

Yet another embodiment of a deodorant device 30 may include a plasma source which does not directly treat a surface of the body, but generates plasma which interacts with a chemical agent which is afterwards applied to a body surface. In this embodiment, the chemical agent is first activated by the plasma inside the deodorant device, wherein the activated agent is then applied to the skin. The chemical agent can be a basic, alkaline, or acidic agent, as well as include a salt tablet or a salty agent.

In still another embodiment, the chemical agent can be adapted to form the dielectric of a SSS source as mentioned above.

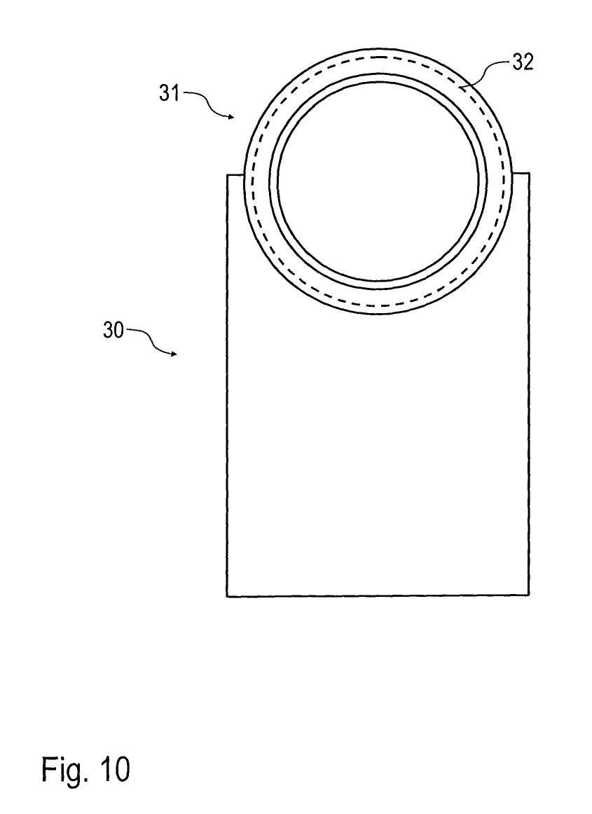

FIG. 10 shows a modification of the deodorant device according to FIG. 8, wherein this embodiment corresponds to the previous embodiment to a large extent. Therefore, reference is made to the above description and the same reference numerals are used for corresponding parts and details.

One characteristic of this embodiment is that the ball-shaped head 31 is in fact rotatable as in conventional deo rollers. In this embodiment, the plasma source is preferably completely integrated into the rotatable ball-shaped head 31. This advantageously avoids the necessity to provide electrical contacts from the lower part of the deodorant device 30 to the rotatable ball-shaped head 31. In a preferred embodiment, switching of the plasma source can be made inductively or by capacitive coupling. Moreover, energy can be supplied to the plasma source inductively or by capacitive coupling. In another embodiment, the rotatable ball-shaped head 31 is supported such as to be slightly movable in a longitudinal direction of the deodorant device 30, preferably being biased in its upper position by an elastic element. If the deodorant device 30 is pressed against a surface to be treated, the head 31 is lowered against the force of the elastic element in its lower position. Thereby, a switch may be activated in order to start plasma generation. The switch is most preferably included by the rotatable ball-shaped head 31 and/or by its support. Also, the outer wall of the head 31 may be slightly deformable, such that plasma generation may be switched on when the wall is slightly deformed.

Preferably, the energy for the plasma source may be gained from a rotating or swivelling motion of the ball-shaped head 31. For this purpose, e.g. an inductive energy harvester may preferably be used. Alternatively, a longitudinal displacement of the ball shaped-head 31 may be used, e.g. by means of a piezo device, an inductive energy harvester or any other suitable means.

In still another embodiment only the at least one electrode is included in the rotatable ball-shaped head 31. The remaining parts of the plasma source are integrated into the lower part of the deodorant device. In this case, an electric connection to the electrodes in the rotatable ball can be made inductively. Also, it is possible to make sliding contacts to the at least one electrode.

Further, the rotatable ball may preferably act as an applicator for a chemical agent as in conventional deodorant devices including a rotatable ball. In this case, the rotatable ball-shaped head 31 is preferably in contact with a reservoir for the chemical agent which is not shown in the figure. The chemical agent distributed over the surface of the ball-shaped head 31 may serve as a dielectric medium such that a plasma source which would function as a SMD source without the agent will work as a SSS source when the agent is applied and covers the outer electrode of the source which faces the surface to be treated. It is also possible that the rotatable ball may be covered with an agent as normally comprised by a deodorant stick. Also in this case, the stick agent or stick material may serve as a dielectric.

Further, the rotatable ball may be replaceable, when the stick material is used up.

Moreover, there is an embodiment of the deodorant device 30, wherein the applicator for the chemical agent is embodied to be a lo stick. The most preferred embodiment includes a ring-shaped stick which encompasses the surface, on which plasma is created. In order to stabilize the stick, it can be encompassed by a wall segment at its outer and/or its inner side. Thus, a breaking or damage of the stick during it application is effectively avoided. The at least one wall segment may be adjustable with respect to its length as measured in a longitudinal direction of the deodorant device. More particularly, if there is an inner and an outer wall segment, these segments may be adjustable independent of each other. The stick may comprise at least the outer electrode facing the surface to be treated. In this case, the material forming the stick, namely the chemical agent, serves as a dielectric medium embedding the electrode.

Another preferred embodiment of the deodorant device includes a protection means like a cap, a lid or a foil which is positioned on the deodorant device at least during transport and most preferably prior to a first use of the device. In a most preferred embodiment, the protective means is reusable such that the deodorant device 30 is not only protective during transport and prior to the first use but also between single events of usage. For example, the protective means can be embodied as a screw cap or a snap-on lid.

Most preferably, the protective means does not only protect the plasma source but also the applicator for the chemical agent. In another embodiment it is also possible to have separate protective means for the plasma source on the one hand and the applicator on the other hand.

In the case where the applicator for the chemical agent includes a reservoir, this reservoir is preferably at least on of refillable and exchangeable. Thus, the deodorant device 30 has not to be disposed only because of an empty reservoir.

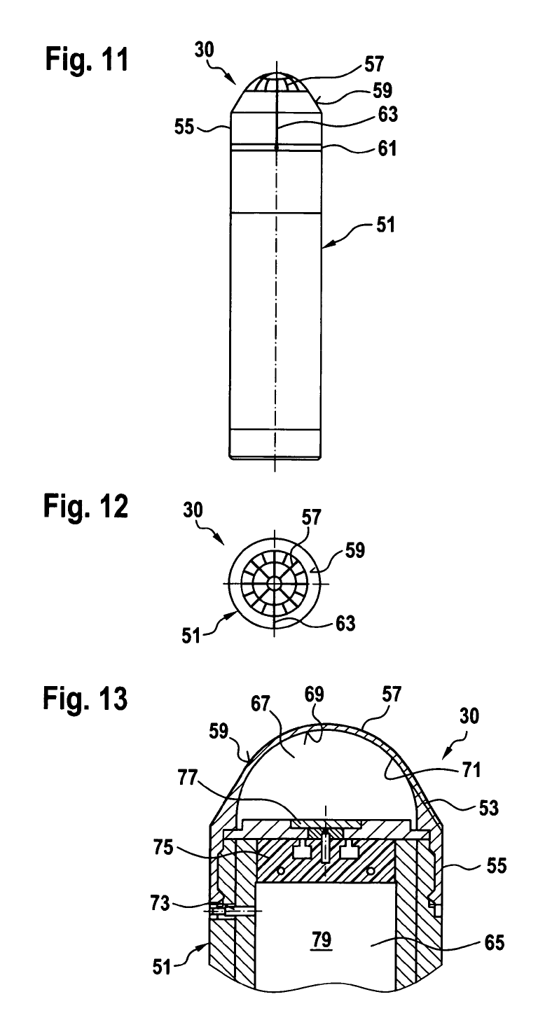

FIG. 11 shows another embodiment of a deodorant device 30 including a plasma source, wherein this embodiment corresponds to the embodiment according to FIG. 8 to a large extend. Therefore, reference is made to the above description and the same reference numerals are used for corresponding parts and details.

The deodorant device 30 depicted in FIG. 11 comprises a housing 51 with a front end 53 being part of the housing 51 of the deodorant device 30. In the embodiment depicted in this figure, the front end has a generally convex shape. Said shape may be varied to a large extend. It is also possible to use a ball shaped front end 53 similar to the embodiment according to FIG. 8 or a flat front end.

The front end 53 has a cylindrical rim which is designed to couple the front end with the rest of the housing 51 which is cylindrical in this case. The housing 51 encloses an interior space of the deodorant device 30 comprising for example among others an electric source for a plasma source of the deodorant device 30.

On top of the front end 53 there is a mesh- or grid-like first outer electrode 57 arranged at the outer surface 59 of the front end 53. The deodorant device 30 comprises a contact ring 61 being electrically connected to the first outer electrode 57 via a conductor 63.

It is clearly to be seen that the first outer electrode 57 is only arranged in the area of the top end of the deodorant device 30. However, it is easily possible to enlarge the first outer electrode 57 in a way that it reaches further down along the outer surface 59 of the front end 53 of the deodorant device 30.

FIG. 12 shows a front view of the deodorant device 30. In this figure the design of the first outer electrode 57 is clearly to be seen. FIG. 12 also shows the conductor 63 which electrically connects the first outer electrode 57 with the contacting ring 61.

The design of the first outer electrode 57 may be amended in a way to adapt the deodorant device 30 to different applications. It is possible to enlarge the first outer electrode 57 to generate more plasma if necessary. It is also possible to amend the design of the first outer electrode 57 to be applicable also to a delicate and sensitive skin of a user of the deodorant device 30.

The front view of the first outer electrode 57 shown in FIG. 12 shows that the electrode comprises three eccentric circles, the centre of which is arranged at the centre of the front end 53. Between the concentric circles regularly arranged parts of the electrode are connecting two or more of the concentric rings. It is clearly to be seen that also meander-shaped lines of conductive material may be used to realize the first outer electrode 57. Additionally, comb-like lines may be arranged on the outer surface 59 of the front end 53 to realize the first outer electrode 57.

The material used for realizing the first outer electrode preferably is chosen to be inert and rust-proof especially against moisture and aggressive fluid.

In FIG. 13 there is a longitudinal cut of the enlarged front end 53 of the deodorant device 30. It is clearly to be seen that the front end is convex. The embodiment shown in FIGS. 11 to 13 has a ball-shaped top end. That is why the upper end of the front end 53 is curved like a spherical segment. In the embodiment the spherical segment is arranged on a conical segment.

The front end 53 is hollow. Its rim 55 is preferably connected to the rest of the housing 51 via a snap-on connection. It is also possible to glue the front end 53 to the rest of the housing 51 to make sure that the deodorant device 30 is moisture-proof closed to protect the plasma source with its circuits and the energy source within the interior space 65 of the housing 51. In case the deodorant device 30 is designed as a disposable the complete housing 51 may be closed moisture-proof.

To enable a user to replace the energy source, at least one end of the housing 51 should be closed by a removable cap. If the front end 53 is glued to the rest of the housing 51, it is possible to provide a removable lid, for example at the opposite end of the deodorant device 30.

In the embodiment of the invention, the front end 53 is designed as a removable cap, to allow an exchange of the front end 53 in case the first outer electrode 57 may be worn or destroyed or in case a user would like to use different electrodes because of a delicate and sensitive skin.

In the embodiment shown in FIGS. 11 to 13, the front end 53 is designed as a hollow cap including a free space 67 and having an inner surface 69 as well as an outer surface 59, carrying the first outer electrode 57. On the inner surface 69 a second inner electrode 71 is provided. It comprises or is made of a conductive material. In a preferred embodiment the inner surface 69 is completely coated with a conductive substance, for example metal. It is also preferred to use a conducting glue placed on the inner surface 69 of the front end 53, which comprises or preferably is made of non-conductive material, especially of plastics, more especially of Teflon because this kind of plastics is characterized by very smooth gliding properties; additionally it only very little irritates the skin of a user. The main aspect of the body of the front end 53 is to be non-conductive. That is why also glass, ceramics or other insulating materials can be used to realize the front end 53 of the deodorant device 30.

In a preferred embodiment of the deodorant device 30 the front end 53 with the first outer electrode 57 and the second inner electrode 71 are produced by injection moulding. In this case different plastic materials are used. Conductive plastic material is used to realize the electrodes 57 and 71, while non-conductive material is used for the body of the front end 53. Using this method to produce the front end 53, it is very easy to realize different shapes for the second inner electrode 71 and especially for the first outer electrode 57. In most cases the second inner electrode 71 covers the whole interior surface 69 of the cap-like front end 53. The first outer electrode 57 may be realized as a grid or a mesh, wherein it is easily possible to realize different shapes of grids and/or meshes to adapt the first outer electrode 57 to different applications. In some cases users may prefer a deodorant device 30 generating more plasma. In other cases the user, having a sensitive and delicate skin, may prefer a deodorant device 30 producing less plasma to not irritate the skin. That is why users may prefer larger or smaller first outer electrodes 53 on the outer surface 59 of the front end 53.

Different designs and dimensions, especially of the first outer electrode 57 may not only be realized by using said injection moulding but also when producing grid- or mesh-like first outer electrodes 57 with other methods mentioned above.

In another preferred embodiment, the first outer electrode 57 may be realized by using thin wires, which are arranged grid- or mesh-like and which are then embedded within plastic material, which will be brought onto the wire preferably using an injection moulding process. A cup-like second inner electrode 71 may then be placed onto the inner side of the front end 53. However, the second inner electrode 71 may also be realized by depositing conductive material like metal or glue on the interior surface of the front end 53.

The first outer electrode 57 comprises or preferably is made of a conductive material. Preferably the outer surface 59 is provided with grooves showing a pattern as it is to be seen in FIG. 12. The grooves are filled with a conductive material. It is possible to deposit a conducting substance within the grooves, for example metal or to fill the grooves with conducting glue or another conducting substance which will stick in the grooves. Also the conductor 63 is made the same way.

In another preferred embodiment the whole outer surface 59 of the front end 53 is coated with a conductive substance. Afterwards the conductive substance is removed from the outer surface 59 and only the grid-like pattern of the first outer electrode 57 and the conductor 63 will remain on the outer surface 59 of the front end 53.

The first outer electrode 57 gets in contact with the contacting ring 61 via the conductor 63. The contact ring 61 is electrically connected to the plasma source 75 arranged within the housing 51. FIG. 13 shows that the contact ring 61 is connected via a contact pin 73 which is electrically connected to the contact ring 61. Preferably the contact pin 73 is pressed against the contact ring 61 by an elastic element. It is easily to be seen that it is only important to achieve an electrical connection between the plasma source 75 and the conductor 63. That is why a little contacting element instead of a complete ring between the contact pin 73 would suffice.

The second inner electrode 71 is connected via a connector, not depicted in FIG. 13, to a connecting plate 77 of the plasma source 75.

From the explanation above it is to be seen that the plasma source 75 comprises a first outer electrode 57 being electrically connected via the connector 63, the contact ring 61 and the contact pin 73 to the plasma source 75. The plasma source also comprises a second inner electrode 71 which is conductively connected to the connecting plate 77 of the plasma source 75. The plasma source 75 is supplied by an electric source 79 arranged in the interior space 65 of the housing 51. The electric source 79 may comprise one or more batteries or accumulators to energize the plasma source 75. The housing encloses additional circuitry, one or more switches and so on to activate the deodorant device 30 if needed. Preferably the output of the plasma source 75 may be adjustable by a user.

Also the embodiment depicted in FIGS. 11 to 13 of the deodorant device 30 may comprise an applicator for applying a chemical agent onto the body surface of a user, e.g. nozzles. Such nozzles are not lo shown in FIGS. 11 to 13, but for example in FIG. 9.

From the description related to FIGS. 11 to 13 it is easily to be seen that the deodorant device 30 may be realized without using any movable parts. Additionally, it is possible to exchange the front end 53 of the housing 51 to replace damaged electrodes or to use different electrodes for more or less sensitive skins or when using different chemical agents together with the deodorant device 30.

Referring to FIGS. 11 to 13 and to the description of the configuration of the deodorant device 30 depicted in said figures, it is clearly to be seen that using the deodorant device 30, plasma will be generated at the front side of the housing 51, i.e. in the area of the first outer electrode 57 of the front end 53. A user may move the deodorant device 30 at a distance to his skin or directly touching the skin with the front end 53 of the deodorant device 30. It is also possible to move the deodorant device 30 at a distance to a shirt of a user. The plasma emanating from the first outer electrode 57 will penetrate the fabric of the cloth of the shirt and will reach the skin of the user if the distance is not too large. Additionally, it is possible to touch the outer surface of the shirt with the front end 53 of the deodorant device 30. In this case the plasma will mainly be generated at the far end of the first outer electrode 57, i.e. within the shirt of the user. It is possible that also in this case, when touching the fabric of a shirt, additionally plasma will be generated at the surface of the first outside electrode 57 and within the fabric of the shirt. This will result in a decontamination and disinfection of the outer surface of the shirt and the shirt itself, while the plasma generated at the far end of the fabric of the shirt will decontaminate and disinfect the skin of the user.

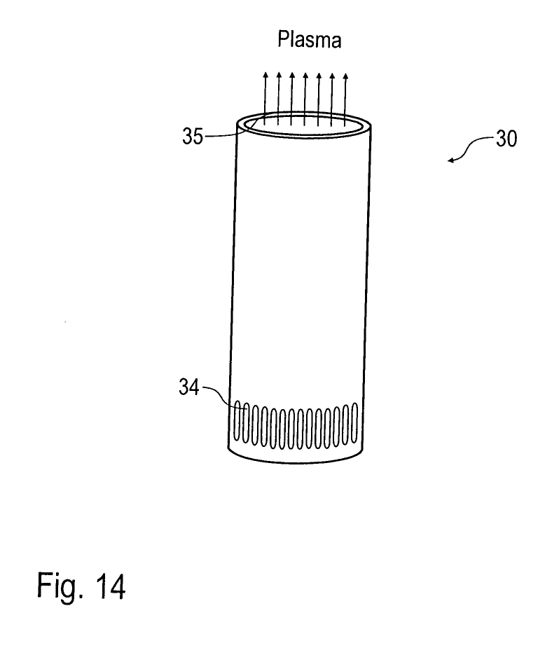

FIG. 14 shows another embodiment of a deodorant device 30 including a plasma source in the form of a so-called plasma jet which draws in ambient air through inlet openings 34 at the bottom of the deodorant device 30, while the low-temperature plasma is applied through an outlet opening 35 at the top of the deodorant device 30.

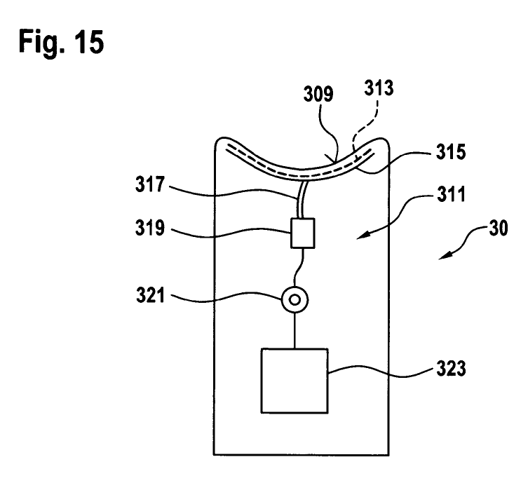

FIG. 15 shows still another embodiment of the deodorant device 30 including a surface 309 on which plasma is generated. The surface 309 has a curved shape, in particular a concave shape in this embodiment as shown here. When the deodorant device 30 is brought in close contact to a surface to be treated, a closed volume is preferably formed, wherein the walls of the closed volume comprise the surface 309 and the surface to be treated. Thus, the treatment of the surface is most efficient, because no external disturbing effects like for example air flow may hinder the plasma treatment within the closed volume. Due to the enhanced efficiency of the treatment, it may be possible to apply a reduced amount of plasma which may be advantageous in particular when treating sensitive surfaces like sensitive skin.

With respect to all embodiments of the invention, it is desirable to realize a closed volume for the plasma treatment of an inner surface thereof, whenever this is possible. Therefore, the appliance according to the invention may preferably comprise a surface, on which the plasma is generated, which is adapted or adaptable, e.g. flexible, to the surface to be treated in order to realize a closed volume for the treatment.

Preferable, the deodorant device 30 comprises a plasma source generally indicated as 311. In the embodiment depicted here, the plasma source 311 includes a first electrode 313 and a second electrode 315. Both electrodes are connected via electrical lines 317 to a power source 319. Preferably, the first electrode 313 is grounded, while a high voltage of alternating current having a suitable frequency for generating plasma is applied to the second electrode 315. Further, the plasma source 311 preferably includes a switch 321 by which generation of the plasma can be started and/or stopped. For example, it is possible that the switch only starts a plasma generation, while the generation stops after a predetermined time interval without another switching event. However, in a preferred embodiment, plasma generation is started by switching the switch 321, and is also stopped by switching the switch 321. There is another preferred embodiment, wherein two switches are provided, a first switch for starting plasma generation, and a second switch for stopping plasma generation.

Moreover, the plasma source 311 preferably includes an energy source 323. In one embodiment, the energy source 323 is adapted to be a battery, an accumulator, a capacitor or another energy storage device. In still another embodiment, the energy source 323 may be external to the deodorant device 30, wherein a connection to the energy source can be made via a plug. In particular, the deodorant device 30 can be mains-driven.

In still another embodiment, the energy source 323 comprises an energy-harvesting device. This can be a piezo crystal, a coil with a magnet movable within the coil, a thermo electric device using the Seebeck effect, Peltier effect, the Thomson effect or another thermoelectric effect. If the energy source 323 comprises an energy-harvesting device, it can preferably include the power source 319. For example, if a piezo crystal drives the plasma source 311, the piezo crystal is preferably adapted to be the energy source 323 as well as the power source 319.

It is emphasized that an application of the deodorant device is not restricted to the human body. The invention also includes a device for sterilizing, decontaminating and/or disinfecting the body of an animal, in particular a pet, wherein the device is especially useful to eliminate sources of animal malodour.

The invention can preferably be embodied as a device for mobile sterilization of surfaces. The term surface thereby includes the fabric of textile materials such as clothing, wherein the plasma penetrates the fabric and does not only act on the immediate surface but develops a certain depth effect. This is also the case with leather surfaces, wherein the plasma penetrates to a certain extent the pores of the leather.

Further, the invention includes a preferably mobile and most preferably hand-held device selected from at least one of a device for reducing itching caused by insect bites, a device for protection against or treatment of athlete's foot and other fungal diseases, a device for reducing tooth ache, a device for the treatment and/or healing of wounds, a device for the treatment or healing of skin irritations, and a device for sterilizing and/or decontaminating food. More particular, the invention includes a device intended for or having use or application in all treatments, cures, preventions, diagnosis of daily familiar wounds, skin irritations and infections, insect bites, foot fungus, acne, herpes, burns, athletes foot, ear infections, diaper/nappy rash, prickly heat, head or body lice and flee, and other invertebrate infestations, dandruff, sensitive skin, nail fungus, psoriasis, cold sores and similar.

Moreover, the invention includes devices intended for or having use or application in masking, modifying, preventing or reducing body odour or other malodour of the person. This includes feet smell, underarm odour, incontinence malodour, intimate malodour and/or internally emanating malodour.

In one embodiment, a device particularly for protection against or treatment of athlete's foot and other fungal diseases includes a stand-alone device or a fixedly installed device, preferably in damp environments, particularly swimming pools and saunas.

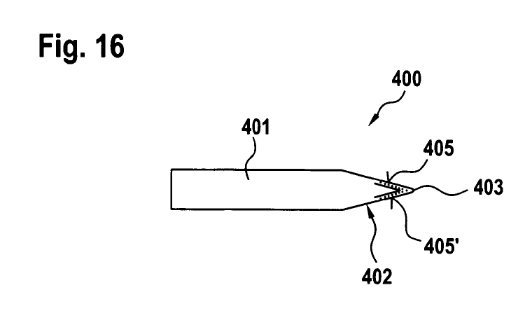

Most preferably, the device is a mobile and/or hand-held device, as generally depicted in FIG. 16 and generally indicated as 400. This embodiment of a mobile device particularly has the form of a stick 401 comprising a plasma source which is only schematically indicated with reference numeral 402.

In particular for cases wherein the device is intended to be used in conjunction with a treatment of geometrically restricted and/or well-defined localized areas, the stick 401 preferably includes a sharp tip 403 for concentrating the plasma within the region of the tip 403. In particular for a treatment of tooth ache or insect bites, a localized application of the plasma is desirable.

However, it is also possible to construct the plasma source 402 in a way such that a greater surface can be used for the treatment. For example, the flanks 405, 405′ of the stick 401 can be used for the treatment of more extended areas, which is in particular desirable in the treatment and/or healing of wounds and the treatment and/or healing of skin irritations. If the stick 401 is cylindrical, the flanks 405, 405′ visible in FIG. 16 may be part of a single circumferential surface.

The tip 403 and the flanks 405, 405′ are also suitable for penetrating the space between individual toes of a foot, such that athlete's foot and other fungal diseases are most effectively treatable by the device.

Further, the invention includes a device which is constructed to be and/or integrated into a nail clipper. For example, the nail clipper may comprise a piezo electric crystal which is activated when a nail is clipped. The plasma source is integrated into the nail clipper in such a way, that the area under the nail which is clipped can be treated, particularly sterilized and/or disinfected simultaneously to, before or after the nail clipping event.

The invention also includes a device which is constructed to be or integrated into a tooth brush. Energy for generating the plasma can be harvested from the tooth brushing movement, or the tooth brush includes a preferably rechargeable energy source. Plasma can be created during the cleaning of the teeth, such that teeth and/or gingiva are disinfected, sterilized and/or decontaminated. Moreover, due to the harmful effect of the plasma to pathogenic germs, a source of malodour of the mouth is eliminated. The device can also be embodied to be suitable for cleaning, sterilizing, decontaminating and/or disinfecting pockets of the gingiva. In this case, periodontitis and gingivitis are effectively treatable with the device. The device is also suitable for the prevention of periodontitis, gingivitis and other diseases of the gingiva.

Further, the invention includes a device which is constructed to be or integrated into a breast pump for sterilizing, decontaminating and/or disinfecting the breast of breast-feeding women.

The invention also includes a device embodied to be or integrated into a shaver. The shaver can be embodied as an electric or a safety razor for wet shaving. The plasma source is preferably arranged such as to sterilize, disinfect and/or decontaminate the razor plates, the parts of the razor which come in contact with the skin and/or the skin of a person using the razor.

Further, the invention includes a device suitable for disinfecting, sterilizing and/or decontaminating food. For example, the device can have the shape of a pepper mill, be embodied to be a pepper mill or be integrated into a pepper mill. Thus, food can be sterilized on the plate in the very moment prior to enjoyment.

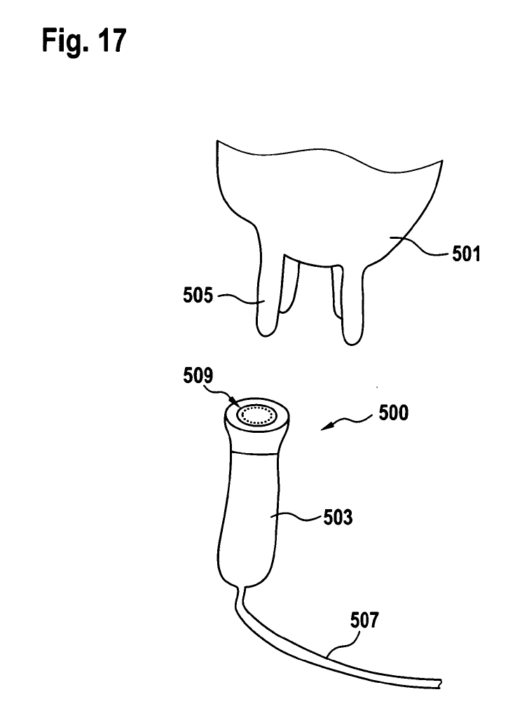

FIG. 17 shows another embodiment of the invention. Generally indicated with reference numeral 500 is a device for disinfecting at least partially an udder of a milkable animal. It is emphasized that the invention is not limited to an application with milkable animals having an udder. It is also possible to treat the teats of milkable animals not having an udder with a device according to the invention.

The device 500 can be embodied to be or integrated into a milking machine. Schematically shown in FIG. 17 is one of typically four suction parts 503 of a milking machine which sucks milk from a teat 505 of the udder 501, wherein the milk is sucked through a tube 507 to a reservoir not shown in the figure. Generally indicated with reference numeral 507 is a plasma source 509 integrated into the suction part 503.

In one embodiment, the plasma source is completely integrated into the suction part 503. In this case, electrical lines may in particular be integrated in or generally follow the tube 507 in order to provide energy to the plasma source 509. However, in another embodiment the plasma source 509 is driven by ambient energy caused by the suction movement of the suction part 503. For example, the plasma source 509 may comprise a piezo electric power source, which is driven by the suction movement which sucks milk out of the teat 505. In still another embodiment, the suction part 503 may include a battery, an accumulator, a capacitor or another energy storage device.

The plasma source 509 is preferably built integral with the suction part 503. However, in another preferred embodiment, the plasma source 509 is constructed as or integrated into an insert usable with the suction part 503. In this case, it is possible to use the plasma source 509 with existing suction parts 503, in particular to upgrade or retrofit existing suction parts 503 with a plasma source 509.

FIG. 18 shows a schematic view of a washing machine 36 including a plasma source 37 for applying a non-thermal plasma to the clothes within the washing machine 36.

It was already mentioned above that the plasma is able to penetrate the fabric of textile materials. In this context, the application also includes a device for sterilizing, disinfecting and/or decontaminating cloths and/or clothing. This device can be built to be a movable or mobile, preferably hand-held device. However, it is also possible to embody the device as a stand-alone device preferably fixedly installed in a laundry. In another embodiment, the device can be included by a machine particularly used in a laundry, such as a washing machine or another laundry appliance like a laundry ironing machine or a mangle. In particular with a mobile device it is possible to sterilize sportswear even during wearing the same, thereby effectively eliminating a source of malodour.

The invention also includes a device adapted to sterilize, disinfect or decontaminate the air which is breathed by a person and/or at least partially surfaces of the mouth and/or the nose. In particular, the device may be adapted to be or integrated into a mask, particularly a surgical mask. In this case, air which is in- or exhaled as well as surfaces of the mouth and the nose are preferably sterilizable with the device.

Another preferred embodiment of the invention includes an appliance which is adapted to be and/or integrated into a device for the cleaning of household, commercial or industrial surfaces, in particular floors. In this case, the device may include a vacuum cleaner or a device for wet cleaning a floor. In particular, the device can be embodied to be a cleaning robot. The device preferably comprises an applicator for applying or delivering a chemical agent to the surface to be cleaned, in particular a cleaning agent.

Moreover, the invention comprises an appliance which is build to be and/or integrated into a catheter, in particular for medical treatment within the scope of minimally invasive surgery or keyhole surgery. Preferably, the catheter comprises a plasma source suitable for sterilizing and/or disinfecting an inner surface of the human or animal body. Therefore, the plasma source can be brought in vicinity of the surface by means of the catheter. Then, the plasma source may be activated in order to plasma treat the surface. For example, it is possible to sterilize and/or disinfect aortic valves with the help of the catheter, in particular prosthetic heart valves.

Further, the invention includes an appliance being embodied to be or integrated into an endoscope. Thereby, a plasma source can be introduced in the body preferably through an existing body opening such as e.g. mouth or anus, in order to sterilize, disinfect and/or decontaminated an internal surface of the body.

Still another embodiment comprises a device which is adapted to be introduced into the mouth in order to sterilize, disinfect and/or decontaminate the inner surfaces of the mouth or in particular the throat. The device may be adapted to be or integrated into a laryngoscope. Thus, a bacterial contamination of the mouth and/or throat is efficiently treatable. In particular, the device is suitable for a treatment of tonsillitis.

Moreover, FIG. 19 shows a schematic side view of a conveyor 38 comprising a conveyor belt 39 and a plasma source 40 which is arranged beneath the upper conveyor belt 39. The plasma source 40 applies a non-thermal plasma through the permeable upper conveyor belt 39 to objects 41 thereby sterilizing the upper surface of the upper conveyor belt 39 and/or the objects 41 on the conveyor belt 39.

FIG. 20 shows a diagram illustrating the on- and off-times of the plasma sources mentioned above. Preferably, the off-time Toff is much longer than the on-time Ton. In this connection, it should be noted that the sterilization/disinfection also occurs during the off-time Toff due to the so-called after-glow effect. This effect means that after the plasma source is switched off, the plasma decays with a certain time constant such that it remains active a certain time on the surface where it is generated. If the off-time TOFF is chosen to be on the order of a time during which the plasma remains active on the surface, there is always a sterilizing effect on the surface for all times t whether the plasma source is switched on or off.

The invention also includes an appliance which is built to be a container or as a device for sterilizing and/or disinfecting the inside of a container, a bottle or a tube.

Thereby, in one embodiment, the container comprises the plasma source preferably as part of a lid of the container.

In another embodiment, the container, the bottle and/or the tube includes a first electrode, wherein a second electrode is preferably arranged outside the container, the bottle and/or the tube. This second electrode is driven by a power supply in order to ignite a discharge between the first and the second electrode. Thus, plasma is formed inside the container, the bottle and/or the tube.

In still another embodiment, the container, the bottle and/or the tube does not comprise any electrode. An external appliance comprising a plasma source is used to generate plasma inside the container, the bottle and/or the tube. Preferably, the appliance comprises a suction means for sucking a flexible wall or a flexible surface of the object to be sterilized onto the surface of the appliance which comprises the plasma source. When the surfaces are close to each other, plasma can be generated inside the container, the bottle and/or the tube. Therefore, the inside of the container, the bottle and/or the tube can be sterilized and/or disinfected and/or decontaminated without any need for the object to be sterilized to comprise an electrode.

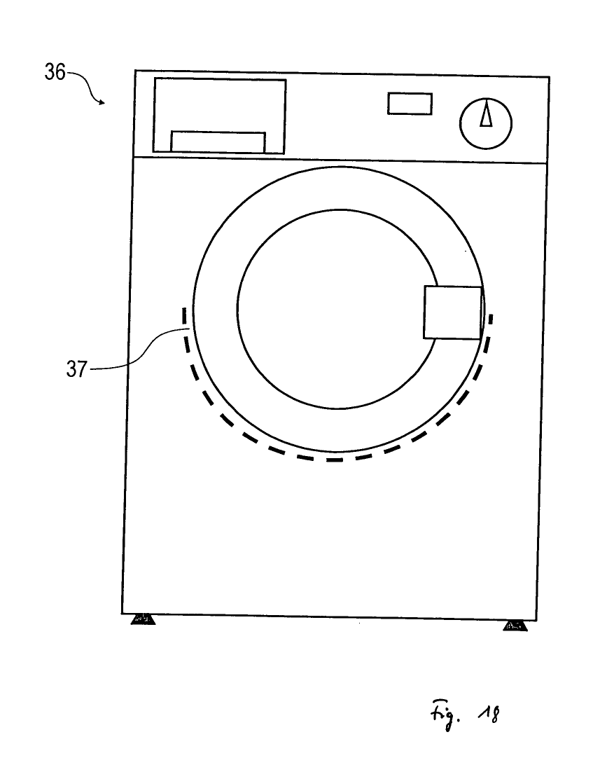

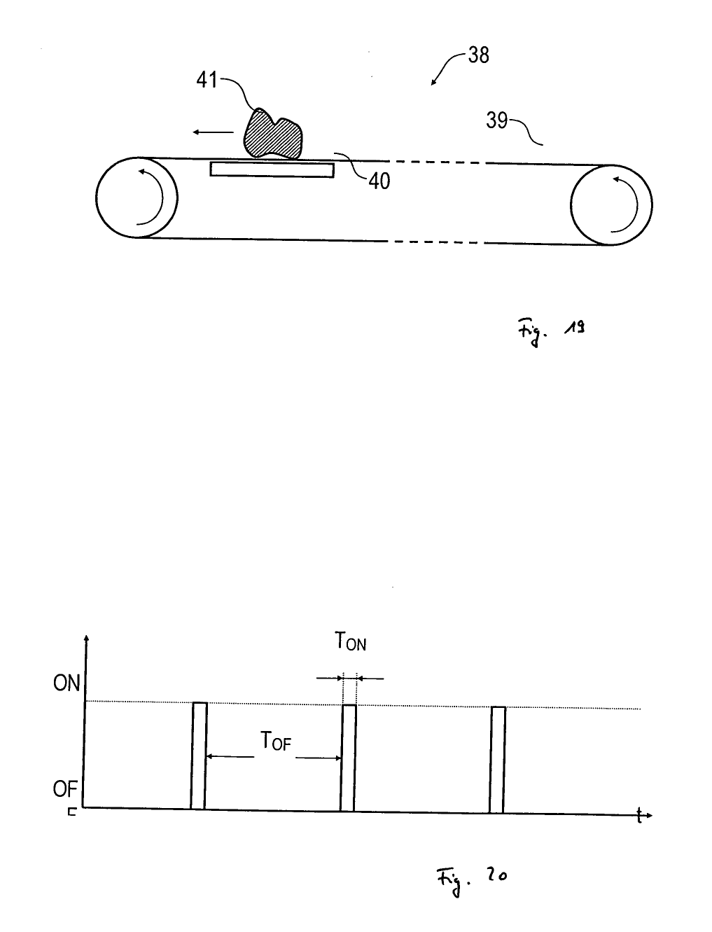

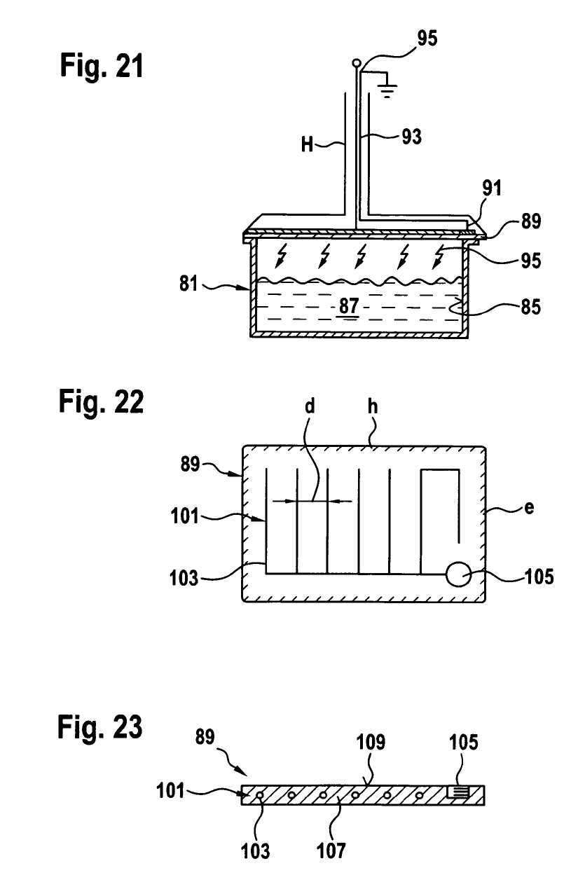

FIG. 21 shows an embodiment wherein the surface to be disinfected and/or sterilized and/or decontaminated is separated from the appliance including the plasma source. In this case, the invention encompasses a container 81 including one or more objects or a substance to be exposed to plasma.

The container 81 comprises a first element, preferably a body enclosing an interior space 85 wherein the objects or substances are placed. In the embodiment depicted in FIG. 8 the container 81 is filled with a substance 87, for example foodstuff or the like.

The container 81 comprises a lid 89 which is placed at the opening of the body 83 to tightly close the container 81. The connection between the lid 89 and the body 83 may be realized by gluing, melting, ultrasonic welding or another known method.

The lid 89 comprises a first electrode which will be explained in relation to the following figures.

FIG. 21 shows that a counter electrode 91 is placed onto the lid 89 and comprises connecting cables 93 connecting the counter electrode 91 with an electric source 95 powering the electrodes to generate plasma 97, indicated by arrows, within the container 81. Preferably cold atmospheric plasma will be generated by the electrodes mentioned above.

The counter electrode 91 preferably is realized as a stamp-like element, especially as a stamp, the counter electrode 91 itself being the basic element of the stamp and having a handle H which is made, as the counter electrode 91 itself, of insulating material. The connecting cables 93 are embedded in the handle H.

FIG. 22 shows a top view onto the lid 89. It comprises a first electrode 101, comprising strip-like conductive areas, for example comb-like arranged wires 103 connected to a contact 105 which is meant to realize a connection to ground.

Preferably, the wire 103 is very thin having a diameter of about 5 μm to 18 μm, preferably of 10 μm to 50 μm.

The distance between two comb-like arranged wires is chosen in a range between 1 mm to 10 mm, preferably between 2 mm to 5 mm.

The wire 103 preferably is embedded within the body of the lid 89 to be protected against damages. However, it is also preferred to realize the first electrode 101 by depositing a conductive material, preferably metal, on the inner surface of the lid 89. The lid itself is made of insulating material, preferably of plastics. In this case preferably the wire 103 can be embedded within the lid, but it is also preferred to deposit a conductive material on the inner surface of the lid 89. It is also preferred to totally cover the inner surface of the lid 89 with a conductive substance and take off all the substance from the inner surface except from the area where the inner electrode is realized.

The contact 105 may be realized the same way, namely by embedding a conductive element, preferably made of metal, within the lid and connecting the wire to it.

The first electrode may preferably be realized by injection moulding the lid using two components. The main part of the lid 89 is made of non-conducive plastic while the first electrode 103 and the contact 105 is made of conductive plastic. It is also preferred to realize notches within the inner surface of the lid 89 having the shape of the inner electrode 101, i.e. being arranged in a comb-like pattern, and to fill the notches with conductive material, for example conductive glue or to deposit a conductive substance within the notches. Also the contact 105 can be realized this way, i.e. by preparing a cavity in the inner surface and to fill it with conductive material.

The pattern of the inner electrode may be varied. For example it is possible to realize two comb-like areas, engaging each other without getting in contact. Also meandric or wave-like first electrodes 101 can be realized in this case. Further, mesh-like structures or arrays are possible.

FIG. 22 shows an outer edge e of the lid 89 and a hachure h indicating the area, where the lid 89 is tightly connected to the body 83 of the container 81.

FIG. 23 shows an enlarged cross section through the lid 89. The insulting basic material 107, the lid 89 preferably is made of, and the wire 103 are clearly to be seen, also the contact 105, being in embedded in the inner surface 109 of the lid 89.

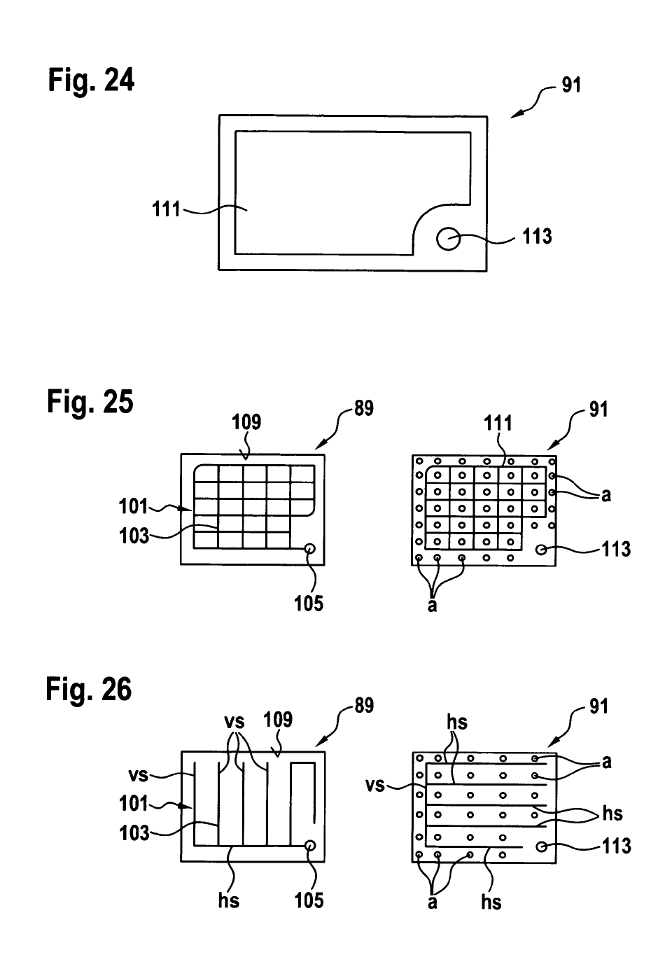

FIG. 24 shows a top view of the counter electrode 91. It is clearly to be seen that the lower surface of said electrode, which will be placed on the lid 81, comprises a second electrode, realized as an conductive area 111 comprising or being made of conducting material. In the embodiment of the counter electrode 91 depicted in FIG. 24 the area 111 is made of a metal layer, preferably of stainless steel. The surface of the counter electrode 91 shown in FIG. 24 also comprises a contact area 113 not being electrically connected to the area 111 and being arranged at an area to be electrically connected to the contact 105 in case the counter electrode 91 is placed on the lid 89, as shown in FIG. 21. The area 111 and the contact area 113 of the counter electrode 91 are connected to the connecting cables 93 and to the electric source 95 shown in FIG. 21.

To activate the plasma 97 within the container 81, the stamp-like counter electrode 91 will be placed onto the lid 89 and the electric source 95 will be activated. Powering the electrodes, the counter electrode 91, i.e. the conductive area 111 acting as a second electrode, plasma 97 will be generated at the first electrode 101 of the lid 89. The plasma disinfects and/or sterilizes and/or decontaminates the content of the container 81, i.e. the free area under the lid 89 and the content, for example the foodstuff 87.

The lid 89 described in relation to FIGS. 21 to 23 is preferably made of a more or less rigid material. The counter electrode 91, also being made of rigid material may easily be brought in contact with the lid 89 using a sufficient pressure to activate the first electrode 101 of the lid 89 when activating the second electrode, i.e. the area 111, of the counter electrode 91 by the electric source 95.

FIG. 25 shows a lid and a counter electrode which are used in case the lid 89 and possibly the container 81 are soft and bendable. Both elements correspond to the embodiment according to FIGS. 21 to 24 to a large extent. Therefore reference is made to the above description, and the same reference numerals are used for corresponding parts and details.

At the left side of FIG. 25 the lid 89 is depicted in bottom view showing the inner surface 109 of the lid 89. At the right side of FIG. 25 the counter electrode 91 is also depicted in bottom view. In case the lid 89 is made of soft and bendable material, the structure of the first electrode 101 and the structure of the area 111, comprising or made of conducting material, are very similar, preferably more or less identical. For example, the first electrode 101 comprises wires 103 running vertically to each other, i.e. a number of wires is arranged horizontally in FIG. 25 and a number of wires is arranged vertically. Accordingly, the conductive area 111 of the counter electrode 91 is realized by vertically and horizontally arranged lines of conductive material. The pattern of the wire of the lid 89 and the pattern of the conductive area 111 of the counter electrode 91 is as similar as possible.

FIG. 25 shows that the counter electrode 91 comprises a number apertures a being connected to at least one suction means applying a negative pressure to the surface of the counter electrode. In the embodiment depicted in FIG. 25, the counter electrode 91 comprises a pattern of apertures a being arranged in vertical or horizontal lines. Preferably, each square between horizontal and vertical lines of conductive material of the conductive area 111 comprises at least one aperture a.

Preferably, also in this embodiment, the counter electrode 91 is realized like a stamp. In case the stamp is lowered to the soft and bendable lid 89 to activate the plasma 95 a suction means is activated to suck the lid 89 against the surface of the counter electrode 91. By this the electrodes of the lid 89 and the counter electrode 91 are arranged in a defined position, preferably parallel to each other. The contact 105 of the lid 89 and the contact 113 of the counter electrode 91 are pressed together to realize a conductive contact between both contacts. Energizing the first electrode 101 and the second electrode, i.e. the conducting area 111 of the counter electrode 91 will result in a generation of plasma 95.

FIG. 26 shows another embodiment of a lid (left-hand) and a counter electrode (right-hand). This embodiment corresponds to the embodiment according to FIG. 25 to a large extent. Therefore reference is made to the above description and the same reference numerals are used for corresponding parts and details.

It is clearly to be seen that the lid 89 and the counter electrode 91 only differ from the embodiment depicted in FIG. 25 by having different electrodes:

The first electrode 101 at the inner surface 109 of the lid 89 is realized as depicted and described in FIG. 22 and comprises strip-like conductive areas, especially realized by a wire 103. That is why reference is made to the description of said Figure. The wire 103 of the first electrode 101 is arranged comb-like having a number of vertical sections vs of wire, said wires being connected to each other by a horizontal section hs of a wire which is connected to the contact 105.

The second electrode, i.e. the conductive area 111 of the counter electrode 91 is realized by a number of horizontal, strip-like areas hs of conducting material, especially wires, being connected by a vertical section vs of a wire or connecting area. Also in this case a comb-like pattern is realized.

From FIG. 26 it is easily to be taken that the comb-like pattern of the first electrode 101 of the lid 89 comprises a number of vertically oriented sections, while the comb-like pattern of the second electrode, i.e. conductive area 111 of the counter electrode 91, comprises a number of horizontally oriented sections. In other words, the conductive strip-like areas of the first electrode 101 realize a first pattern, wherein the conductive sections are oriented in a first (vertical) direction, while the conductive strip-like areas of the second electrode, i.e. conductive area 111, of the counter electrode 91, realize a second pattern, wherein the conductive sections are oriented in a second (horizontal) direction. The strip-like areas of the first and second electrodes include an angle a of preferably 90°. This embodiment is shown in FIG. 26. The angle between said strip-like areas may be chosen in a range of 0°<α<180°, preferably of 45°≦α≦135°.

Also in this embodiment, the counter electrode 91 comprises apertures a as mentioned above and a contact area 113, as described in relation to FIG. 24.

To generate plasma 95 within the container 81 the—preferably stamp-like—counter electrode 91 is lowered onto the upper surface of the lid 89 and a pump or the like is activated to suck air through the apertures a. This will result in sucking the soft and bendable lid 89 against the counter electrode 91.

At the crossing areas of the first electrode 101 and the conductive area 111 of the lid and the counter electrode 91, plasma will be generated if the electric source 95 is activated.

The embodiment shown in FIG. 26 will allow to generate plasma 95 in case it is not possible to realize very similar electrodes (first electrode 101 of the lid 89 and conducting area 111 of the counter electrode 91) as shown and described in FIG. 25.

It is clearly to be seen that it is possible to generate plasma within a container 81 comprising a mostly solid and stable lid 89 as described in relation to FIGS. 21 to 24. However, it is also possible to generate plasma 95 within a container 81 which is tightly closed by a soft and bendable lid as described in relation to FIGS. 25 and 26.

When filling a container 81 with objects or a substance, especially foodstuff 87 or medical substances, preferably first of all the container 81 will be disinfected and/or sterilized and/or decontaminated, especially with plasma, before filling it. After filling said container 81 the lid 89 will be firmly, especially tightly connected to the body 83 of the container 81. Then the counter electrode 91 will be placed onto the lid 89. In case it is a stable lid, the first electrode 101 of the lid 89 and the second electrode, i.e. the conducting area 111 of the counter electrode 91 are arranged closely to each other in a defined distance.

In case of soft and bendable lids 89, as described in relation to FIG. 25 and 26, the electrodes may not be arranged close enough to each other to generate plasma after activating the electric source 95. That is why it will be necessary in this case, i.e. using soft and bendable lids, to activate a pump or other mean to suck air through the apertures a of the counter electrode 91 and to suck out the air between the counter electrode 91 and the lid 89. By this the electrode 101 of the lid 89 and the conducting area 111 of the counter electrode 91 will be arranged closely to each other. It is now possible to generate plasma within the container 81, a bottle or a tube. The tube may be a shrink tube, in particular a heat shrinkable tube, in a preferred embodiment.

Generally, it is only necessary to activate the power source 95, for example for 2 to 10 seconds. Even after switching off the power source 95 there will be an after glow within the container 81 resulting in a continued disinfection and/or sterilization and/or decontamination of the interior space 109 of the container 81 and its content.

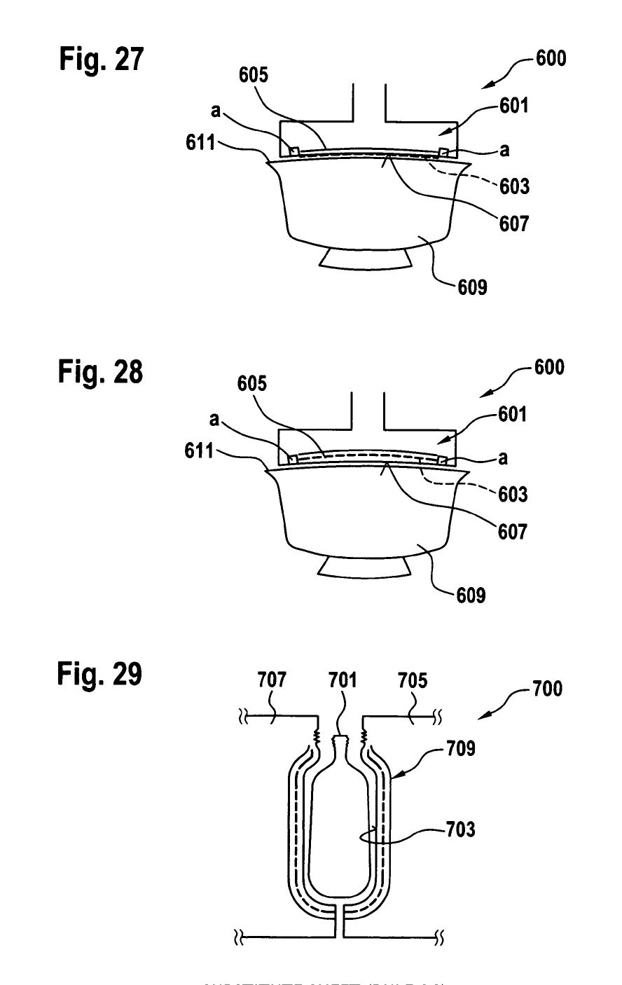

FIG. 27 shows a schematic view of another embodiment of the present invention. The embodiment according to FIG. 27 comprises an appliance generally indicated with reference numeral 600 which comprises a plasma source generally indicated with reference numeral 601. The plasma source 601 comprises a first electrode 603, and a second electrode 605. The first electrode 603 is arranged at a surface 607 of the appliance 600, which faces an object to be treated. In the example depicted in FIG. 27, the object to be treated is a container 609, e. g. a carton for yoghurt or blancmange, or pudding, respectively. The container 609 comprises a flexible or bendable lid 611.

In contrast to the embodiment shown in FIGS. 21 to 26, the lid 611 does not comprise any electrode. Instead, the first and the second electrode 603, 605 are arranged external to the container 609 and integrated into the appliance 600 which is preferably embodied as a stamp. At least the second electrode 605 is embedded in a dielectric material.

In the embodiment shown in FIG. 27, the first electrode 603 is accessible and/or exposed at the surface 607. Thus, it makes direct contact to the lid 611. This means, that preferably the plasma source 601 realises the principle of a surface micro discharge (SMD) plasma source.

Preferably, the appliance 600, in particular the surface 607, includes apertures a, two of which are schematically shown in FIG. 27. These are in fluid connection with at least one suction means as described above in conjunction with FIGS. 25 and 26. Thus, the apertures a are adapted and arranged in order to suck a flexible wall or a flexible surface of the object to be sterilised, here the lid 611 of the container 609, onto the surface 607 of the appliance 600. Therefore, air is sucked through the apertures a in order to perform the sucking action.

However, the appliance 600 does not necessarily include at least one suction means and apertures a. If the object to be sterilised has a wall segment, surface or lid which is rigid enough, appliance 600 can just be pressed with its surface 607 onto the object to be sterilised in order to perform the sterilising action.

However, in the case that a suction action should be performed, surface 607 is preferably at least slightly curved, as indicated in FIG. 27. Thus, a flexible wall segment or lid 611 can be sucked most efficiently onto the surface 607. A close contact between the surface 607 and the lid 611 is then guaranteed.

The object to be sterilised is not necessarily a container 609. In another embodiment, a tube can be sterilised with appliance 600. In this case, surface 607 is adapted to encompass the tube, and an outer wall segment of the tube is preferably sucked against the surface 607, which has most preferably an annular shape. In still another embodiment, the object to be sterilised is a bottle, wherein the appliance 600 is adapted to the shape of the bottle at least in the region of the surface 607.

What is said in the following about the container 609 is also true for a bottle and/or a tube as the object to be sterilised. Only for clarity's sake, the explanations which follow are made in conjunction with the container 609 as depicted in FIGS. 27 and 28. In this context, the lid 611 corresponds to a wall segment of the bottle and/or the tube of the embodiments not shown in FIGS. 27 and 28.

In one embodiment, the lid 611 comprises a dielectric material. In this case, when the lid 611 is in close contact to the surface 607, the exposed first electrode 603 is covered by the dielectric material of the lid 611. Thus, the surface micro discharge plasma source of the appliance 600 forms a plasma source realising the principle of a self-sterilising surface together with a dielectric material of lid 611. In total, plasma is generated on the side of lid 611 facing the inside of the container 609, thereby sterilising at least the inner surface of the lid 611 and most preferable the inside of the container 609.

In another embodiment, the lid 611 is made from a conductive material, e.g. metal, particularly a metal foil. In this case, the exposed or accessible electrode 603 and the lid 611 will have the same potential when they are in close contact. Thus, a discharge will be created between the second electrode 605 and the lid 611. Therefore, the plasma source 601 together with the lid 611 realises the principle of a surface micro discharge plasma source. Also in this case, plasma is generated inside the container 609.

FIG. 28 shows a schematic view of another embodiment of the invention. Reference is made to the above description and the same numerals are used for corresponding parts and details. In contrast to the embodiment as shown in FIG. 27, the embodiment shown in FIG. 28 has a first and second electrode 603, 605, which are both embedded in a dielectric material. Thus, the first electrode 603 is not accessible or exposed at the surface 607. Instead, the surface 607 is preferably formed by the dielectric material in which at least the first electrode 603 is embedded. Thus, the appliance 600 and in particular the plasma source 601 realises the principle of a self-sterilising surface.

If the lid 611 is made from a dielectric material, and it is sucked or pressed close to the surface 607, the plasma source 601 still realises the principle of a self-sterilising surface, wherein the distance of the surface on which the plasma is generated from the first electrode 603 is increased by the thickness of the lid 611. However, also in this case plasma is generated on the inner surface of the lid 611 supposed it is not too thick. As explained in conjunction with FIG. 27, the plasma generated inside the container 609 sterilises, disinfects and/or decontaminates at least the inner surface of the lid 611 and most preferably the inside of the container 609.

The appliance 600 is preferably applicable to blister packs for medical tablets and/or capsules. As explained above, this is the case whether the blister pack is closed by a dielectric foil or a metal foil.

In still another embodiment, the container to be sterilised with the appliance 600 is first closed with a permeable foil or lid and then sterilised by appliance 600. Afterwards, the container is permanently closed with a metal cap or metal foil or another suitable permanent lid.

Further, the invention includes an embodiment, wherein the inside of a tub which does not have a lid of its own, is sterilised by the appliance 600. In this case, the surface 607 of the appliance 600 serves as a temporary lid for the tub thereby closing the inner volume. The plasma is then generated at the surface 607 of the appliance 600, thereby sterilising the inside of the tub. Afterwards, the appliance 600 is removed, and the tub is again open. However, its inside has been effectively sterilised, disinfected and/or decontaminated.

Moreover, the invention includes a box suitable to sterilize, disinfect and/or decontaminate objects contained within the box. A plasma source is preferably included in a lid of the box as described above. In one preferred embodiment, the lid may be fixed to the box with at least one hinge, such that the lid may be swung open. Preferably, a cloth, in particular a cleaning cloth, can be sterilized, decontaminated and/or disinfected in the box, most preferably prior to cleaning.

FIG. 29 shows a schematic view of another embodiment of the application. In this case, the invention includes an appliance 700 for sterilising a bottle 701. The appliance 700 has a side 703 facing the bottle and preferably following the shape of the bottle for making close contact to an outer surface of the same. In particular, the appliance 700 may comprise two stamp-like elements 705, 707 for preferably completely encompassing the bottle 701. A plasma source 709 included by the appliance 700 is thus brought in close contact to the bottle 701 in order to sterilise the inside thereof according to one of the working principles as explained in conjunction with FIGS. 27 and 28.

Preferably, the appliance 700 is embodied as a tool for forming the bottle 701. In this case, forming the bottle 701 and sterilising its inside can be carried out in one single step.

However, in another embodiment, appliance 700 can be a stand-alone device which can be easily included in an existing plant for making and filling bottles.

Preferably, the bottle 701 is made of plastics, most preferably polyethylene. In this case, the bottle 701 is formed, sterilised and filled in one single filling line.

Advantageously, the plasma source 709 of appliance 700 generates plasma immediately on an inner surface of the bottle 701. In this case, hot electrons are generated directly on the surface which—in addition to the bactericidal and harmful effect of the plasma on pathogenic germs—also dissociate molecules deposited on the surface. In particular, molecules which are responsible for a bad smell or taste of a liquid filled afterwards into the bottle 701 can be effectively dissociated by the hot electrons.

If the appliance 700 is embodied to be integrated into a device for forming the bottle 701, there is abundant time for the after glow effect between a forming of the bottle 701 and a filling of the same in the filling line. Thus, the filling line needs not to be slowed down in order to have the inside of the bottle 701 effectively sterilized, disinfected and/or decontaminated.

In total, sterilizing the bottle 701 in the filling line with the appliance 700 serves to save a huge amount of water which is otherwise needed in order to sterilize the bottle prior to filling. Thereby, the appliance 700 also helps to reduce the costs of running the filling line dramatically.

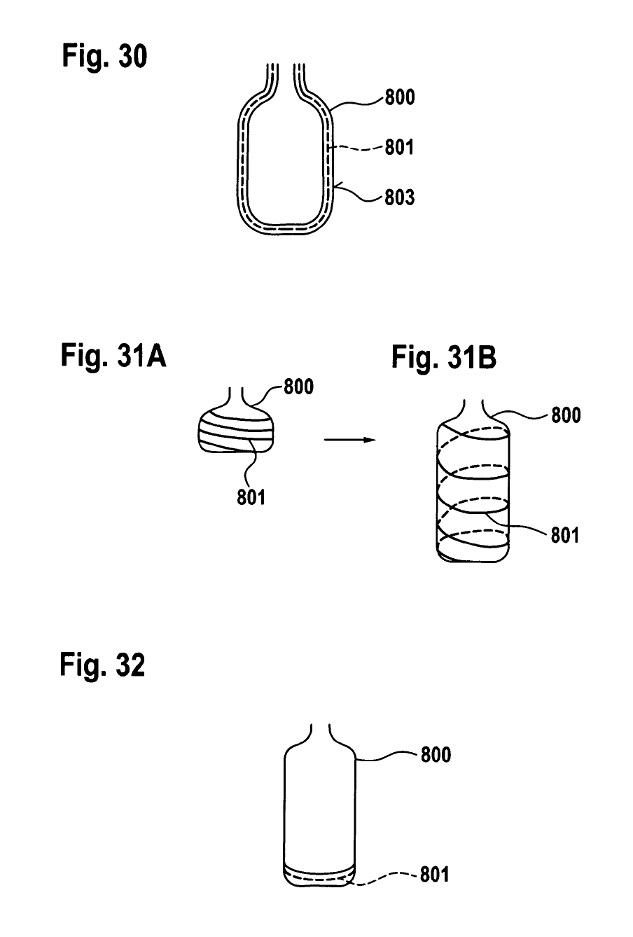

FIG. 30 shows another embodiment of the invention. The appliance is embodied here as a bottle 800. In another embodiment, the appliance is embodied as a tube which is not shown in FIG. 30. At least a wall segment, preferably the complete wall—as in the example of FIG. 30—of the bottle 800 or the tube comprises a first electrode 801. Preferably, the electrode 801 is embedded in the wall segment of the bottle 800 or the tube.

The appliance further comprises a second electrode which is preferably arranged outside the bottle and/or the tube, which is not shown in FIG. 30. This second electrode is driven by a power supply in order to ignite a discharge between the first and the second electrode such that plasma is formed inside the bottle and/or the tube. Therefore, the second electrode is most preferably brought in close contact at least partially to an outer surface 803 of the bottle 800 and/or the tube.

The appliance may also include a pipe or a complete pipeline. In particular, a tube or a pipe can be sterilized in a single region along its extension. However, it is also possible to move the second electrode along the extension of the tube or pipe in order to sterilize the complete inside thereof.

Further, the invention includes a tube which is adapted as a catheter or an endoscope, in particular for medical treatment within the scope of minimally invasive surgery or keyhole surgery. In this case, the inside of the catheter or endoscope may be effectively sterilized and/or disinfected by generating a plasma inside the catheter or the endoscope as described above for the tube.

Moreover, the application includes a tube which is embodied as a shrink tube, in particular a heat shrinkable tube, preferably made from PO (Polyolefin). The inside of the shrink tube is sterilizable according to the invention.

FIG. 31 shows a schematic view of another embodiment of the bottle 800. In this case, in FIG. 31A there is shown a blank of the bottle 800 already comprising the electrode 801. In order to form the bottle 800 which is shown in FIG. 31B, the blank is blown up or vacuum-sucked into the final shape of the bottle 800.

As can be seen from FIG. 31, the electrode 801 substantially follows the shape of a spiral. Thus, when the blank is brought into the final shape of the bottle 800, the electrode 801 can at least be extended along a longitudinal axis of the bottle 800.

However, in reality the blank is not only blown up in a longitudinal direction, but also in a radial direction. Therefore, deviating from FIG. 31, the electrode 801 may not only comprise a substantially spiral shape, but also follow some kind of zig-zag course along its length extension. Thus, the electrode 801 is not only able to axially expand, but also to radially expand by straightening the zig-zag course, thereby preferably forming a substantially straight line along the length extension of the spiral.

In another embodiment, an electrode is only integrated in a part of the bottle which comprises the thread for the closure and the holding element for further processing of the bottle within the filling line. This part of the bottle is made as such from beginning and not changed when the blank is brought into the form of the final bottle. Therefore, the electrode is not affected by the forming step, because it has not to change its geometry, extension or shape. However, since the plasma generated inside the bottle has the general tendency to rise due to its thermal properties, it is difficult if not impossible to sterilize the whole inside of the bottle in the case that the electrode is only integrated in the upper part comprising the thread for the closure. Thus, in this case the bottle has to be turned over prior to sterilization, such that the upper part with the thread becomes the lower part, and the bottom of the bottle becomes the upper part. When generating the plasma under these circumstances at the part of the bottle comprising the thread, the plasma will rise to the bottom, thereby sterilizing the complete inside of the bottle, because it is turned up-side down.

FIG. 32 shows still another embodiment of the invention. In this case, the electrode 801 is only integrated in the bottom of the bottle 800. This has the advantage that the bottle has not to be turned up-side down in order to sterilize its inside. Plasma which is generated on the bottom will rise to the upper part, thereby sterilizing the complete inside of the bottle 800.

It is clearly to be seen that the basic idea of disinfecting, sterilizing and/or decontaminating an appliance may easily be used in generating plasma within a container 81.

Generally, the invention also includes methods for applying the appliances and/or devices as described above. The methods are not explicitly described but clear from the functions and features of the respective devices and/or appliances, and their respective purposes and fields of application.

Although the invention has been described with reference to the particular arrangement of parts, features and the like, these are not intended to exhaust all possible arrangements of features, and indeed many other modifications and variations will be ascertainable to those of skill in the art.

### LIST OF REFERENCE NUMERALS

1 Plasma source

2 Electrode arrangement

3 Housing

4 Driver circuit (high voltage power supply)

5 Connection cable

6 Appliance

7 Surface of the appliance

8 Surface of the plasma source

9 Electrodes

10 Dielectric layer

11 Back electrode

12 Electrode finger

13 Electrode finger

14 Electrode

15 Switching element

16 Control device

18 Kitchen block

19 Sink

20 Ceramic stove top

21 Workplate

22 Laboratory table

23 Workplate

24 Toilet seat

25 Plasma source

26 Escalator

27 Moving handrails

28 Surface of the handrails

29 Plasma source

30 Deodorant device

31 Ball-shaped head

32 Plasma source

33 Nozzles

34 Inlet openings

35 Outlet opening

36 Washing machine

37 Plasma source

38 Conveyor

39 Conveyor belt

40 Plasma source

41 Objects

GND Ground

51 Housing

53 Front end

55 Rim

57 First outer electrode

59 Outer surface

61 Contact ring

63 Conductor

65 Interior space

67 Free space

69 Inner surface

71 Second inner electrode

73 Contact pin

75 Plasma source

77 Connecting plate

79 Electric source

81 Container

83 Body

85 Interior space

87 Foodstuff

89 Lid

91 Counter electrode

93 Connecting cables

95 Electric source

97 Plasma

101 First electrode

103 Wire

105 Contact

107 Basic material

109 Inner surface

111 Area

113 Contact area

301 Applicator

303 Reservoir

305 Pumping device

307 Pipe/tube

309 Surface

311 Plasma source

313 First electrode

315 Second electrode

317 Electrical lines

319 Power source

321 Switch

323 Energy source

400 Device

401 Stick

402 Plasma source

403 Tip

405 Flank

500 Device

501 Udder

503 Suction part

505 Teat

509 Plasma source

600 Appliance

601 Plasma source

603 First electrode

605 Second electrode

607 Surface

609 Container

611 Lid

700 Appliance

703 Side

705 Stamp-like element

707 Stamp-like element

709 Plasma source

800 Bottle

803 Outer surface

H Handle

a Apertures

d Distance

e Outer edge

h Hachure

vs Vertical sections

hs Horizontal sections

1. An appliance for at least partially disinfecting/sterilizing a contaminated surface, comprising an integrated plasma source adapted generate a non-thermal plasma on the surface for at least partially disinfecting/sterilizing the surface by reducing a concentration of pathogenic germs on the surface.

2. The appliance according to claim 1, wherein the surface to be sterilized is a surface of the appliance which is contaminated during use of the appliance.

3. The appliance according to claim 1, wherein
a) the plasma source is a surface micro-discharge plasma source comprising several electrodes, and/or
b) there is a uniform distance between the adjacent electrodes of different polarity, or
c) there is a spatially variable distance between the adjacent electrodes of different polarity.

4. The appliance according to claim 3, wherein the surface micro-discharge plasma source is embedded into a surface of the appliance, so that the non-thermal plasma is generated on top of the surface of the appliance.

5. The appliance according to claim 4, wherein
a) the electrodes of the surface micro-discharge plasma source have a shape which resembles the shape of the surface of the appliance, and/or
b) the electrodes of the surface micro-discharge plasma source are flat and/or planar.

6. The appliance according to, wherein the electrodes of the surface micro-discharge plasma source are arranged in the same plane.

7. The appliance according to claim 6, wherein
a) the electrodes are finger-shaped intertwining each other from opposite directions, or
b) the electrodes are spiral-shaped intertwining each other, and/or
c) the electrodes comprise interlocking branches or kinks.

8. The appliance according to claim 3, wherein
a) electrically opposite electrodes of the surface micro-discharge plasma source are arranged in separate adjacent electrode layers, and/or
b) each of the electrode layers is planar and the separate electrode layers are arranged coplanar relative to each other, and/or
c) the electrodes are switched at a constant or variable frequency.

9. The appliance (6; 18; 22; 24; 26; 38) according to claim 1, wherein
a) the surface to be sterilized is substantially planar, and/or
b) the plasma source (1; 25; 37; 40) comprises a substantially planar surface which is flush with the surface which is to be sterilized.

10. The appliance according to claim, 1, wherein the appliance is
a) water-proof,
b) dust-proof,
c) air-borne particles proof, and/or
d) easy to clean.

11. The appliance according to claim 3, wherein the surface comprise: (a) a corrosion resistant material, which is optionally a member selected from the group consisting of ceramics, glass, glass-ceramics, or (b) a flexible corrosion resistant material, which is optionally a member selected from the group consisting of silicone, Makrolon, and POM.

12. The appliance according to claim 1, wherein the appliance is
a) a work plate, optionally on a kitchen table or on a laboratory table, or
(a) a work plate, optionally on a kitchen table or on a laboratory table, or
b) a cutting board for cutting objects, which is optionally foodstuffs, or
c) a handle, which is optionally a door handle, or
d) bathroom equipment, which is optionally a toilet seat, or
e) a deodorant device for deodorizing and/or disinfecting a body surface, or
f) a moving handrail of an escalator or a moving walkway, or
g) gym equipment, which is optionally a bench or a seat of a training machine, or
h) a device for mobile sterilization of surfaces, or
i) a device for reducing itching caused by insect bites, which is optionally, in a form of a stick comprising the plasma source, or
j) a device for protection against or treatment of athletes' foot and other fungal diseases, optionally adapted to be a mobile device or installed optionally in damp environments, optionally in swimming pools and saunas, or
k) a device for reducing tooth ache, or
l) a device for the treatment and/or healing of wounds, or
m) a device for the treatment or healing of skin irritations, or
n) suitable for disinfection of baby bottles, pacifier, toys, dentures, tooth brushes, hair brushes, or
o) a dishwasher or a dryer or
p) a washing machine, or
q) a conveyor comprising a conveyor belt, wherein the plasma source is arranged in a vicinity of the conveyor belt so that the plasma sterilizes objects which are conveyed on the conveyor belt, or
r) a device for disinfecting hands, or
s) a container, or
t) a device for sterilizing and/or disinfecting an inside of a container, a bottle or a tube, which is optionally shrink tube, or
u) a device for disinfecting at least partially an udder of a milkable animal, or
v) a catheter, or
w) an endoscope, or
x) a shopping cart.

13. The appliance according to claim 12 in a form of a moving handrail of an escalator or a moving walkway, wherein
a) the plasma source is integrated into the moving handrail, so that the plasma source moves with the handrail, or
b) the plasma source is stationary and arranged close to a surface of the handrail, so that the non-thermal plasma generated by the plasma source at least partially disinfects or sterlizes the surface (28) of the handrail.

14. The appliance according to claim 12, wherein the appliance is a deodorant device, comprising an applicator for applying or delivering a chemical agent, preferably at least one of a deodorant, an antiperspirant, and a fragrance.

15. The appliance according to claim 14, wherein applicator for the chemical agent is at least one member selected from the group consisting of a rotatable ball, a spray, and a stick.

16. The appliance according to claim 14, wherein the applicator for the chemical agent is a rotatable ball, and wherein
a) the plasma source is integrated into the rotatable ball, or
b) the plasma source is arranged stationarily outside the rotatable ball.

17. The appliance according to claim 14, wherein the applicator for a chemical agent is a spray, comprising at least one nozzle for spraying an agent onto the surface, wherein optionally the agent interacts with the non-thermal plasma thereby improving the disinfecting or sterilizing effect of the non-thermal plasma.

18. the appliance according to claim 14, further comprising a front end, the front end comprising a first outer electrode of the plasma source and a second inner electrode of the plasma source.

19. The appliance according to claim 18, wherein the front end has a curved shape, which is optionally a convex shape, a concave shape, or shape of a spherical segment.

20. The appliance according to claim 18, where the front end comprises or is made of a non-conductive material, which is optionally PTFE or another plastic.

21. The appliance according to claim 18, wherein the first outer electrode and/or the second inner electrode are/is realized by deposition of a conducting substance on the inner and/or outer surface of the front end, wherein the conductive substance is metal or a conducting plastic or a conducting glue.

22. The appliance according to claim 21, wherein the front end and its first and/or second electrodes are realized by injection molding different materials, optionally plastic materials, wherein conductive material is used for the electrodes and non-conductive material is used for the front end.

23. The appliance according to claim 18, wherein
(a) the first outer electrode and/or the second inner electrode are;
(i) finger-shaped intertwining each other from opposite directions, or
(ii) spiral-shaped intertwining each other, and/or
(iii) comprise interlocking branches or kinks, and/or
(b) electrically opposite electrodes of the surface micro-discharge plasma source are arranged in separate adjacent electrode layers, and/or
(c) each of the electrode layers is planar and the separate electrode layers are arranged coplanar relative to each other, and/or
(d) the electrodes are switched at a constant or variable frequency. separate

24. the appliance according to claim 18, wherein the inner and/or outer surface of the front end comprise/comprises a number of grooves receiving a conductive material to realize the first outer electrode and/or the second inner electrode.

25. The appliance according to claim 18, wherein the front end is a removable cap.

26. The appliance according to claim 25, wherein a number of removable caps is provided having different embodiments of electrodes, optionally first outer electrodes.

27. The appliance according to claim 12 in a form of a mobile device, wherein the device is at least one of rechargeable, battery operated, mains-driven, and driven by an energy-harvesting device.

28. The appliance according to claim 12 in a form of a dishwasher or a dryer, wherein the plasma source is integrated in walls of a housing of the appliance.

29. The appliance according to claim 12, wherein the appliance is a container comprising a body, a lid and a counter electrode with a conductive area, wherein the lid comprises a first electrode and wherein the counter electrode is connected to an electric source.

30. the appliance according to claim 29, wherein the first electrode of the lid and/or the conductive area of the counter electrode comprises conductive material or is made of it.

31. The appliance according to claim 29, wherein the counter electrode is realized separately from the lid and optionally as a stamp-like element.

32. the appliance according to claim 29, wherein the counter electrode comprises apertures (a) connected to a suction device, sucking air through the apertures (a).

33. The appilance according to claim 29, wherein the first electrode of the lid comprises strip-like conductive areas, optionally a wire, arranged in a first pattern, and wherein the conductive area of the counter electrode is realized as a continuous area of conductive material.

34. The appliance according to claim 29, wherein the first electrode of the lid comprises a strip-like conductive area, optionally a wire arranged in a first pattern, and wherein the conductive area of the counter electrode is realized in a similar, or in an identical pattern.

35. the appliance according to claim 29, wherein the first electrode of the lid comprises conductive strip-like areas, optionally a wire, and realizes a first pattern, wherein the conductive sections are oriented in a first direction, and wherein the conductive area of the counter electrode comprises strip-like conductive sections and realizes a second pattern, wherein the conductive sections are oriented in a second direction, and wherein the strip-like areas of the first pattern and the strip-like area of the second pattern include an angle chosen in a range of 0°<α<180°.

36. The appliance according to claim 12, comprising a first electrode and a second electrode, at least the first electrode being arranged at a surface facing an object to be treated, the surface optionally comprising apertures (a) connected to a suction device, sucking air through the apertures (a).

37. The appliance according to claim 36, wherein the suction device and/or the apertures (a) are adapted and arranged to suck a flexible wall or a flexible surface of an object to be sterilized onto the surface of the appliance which comprises the apertures (a).

38. The appliance according to claim 36, wherein the first electrode is embedded in a dielectric material.

39. The appliance according to claim 12, wherein the appliance is embodied as at least one of a container, a bottle, and a tube, which is optionally a shrink tube, wherein at least a wall segment of the at least one of a container, a bottle, and a tube comprises a first electrode.

40. The appliance according to claim 39, further comprising a second electrode, optionally arranged outside the at least one of a container, a bottle, and a tube, the second electrode being driven by a power supply in order to ignite a discharge between the first and the second electrode such that plasma is formed inside the at least one of a container, a bottle and a the tube.

41. The appliance according to claim 1, wherein the pathogenic germs comprise
a) bacteria,
b) spores,
c) viruses,
d) fungi,
e) prions,
f) biofilms comprising any of the afore-mentioned pathogenic germs,
g) microorganisms,
h) allergens,
i) molecules causing inconvience, disturbance and/or debilitation, and/or
j) air-borne particles, which are optionally pollen.
